(19) United States
(12) Patent Application Publication
(10) Pub. No.: US 2026/0041897 A1
(43) Pub. Date: Feb. 12, 2026

(54) TARGETED DRUG DELIVERY METHODS USING A MICRONEEDLE

(71) Applicant: Georgia Tech Research Corporation, Atlanta, GA (US)

(72) Inventors: Jae Hwan JUNG, Atlanta, GA (US); Mark R. PRAUSNITZ, Atlanta, GA (US)

(21) Appl. No.: 19/094,522

(22) Filed: Mar. 28, 2025

Related U.S. Application Data
(63) Continuation of application No. 18/802,027, filed on Aug. 13, 2024, now abandoned.
Application No. 18/802,027 is a continuation of application No. 16/609,583, now Pat. No. 12,090,294.
(60) Provisional application No. 62/500,209, filed on May 2, 2017.

(51) Int. Cl. A61M 37/00; A61K 9/00; A61K 9/16; A61K 31/728; A61P 27/02

(52) U.S. Cl. CPC A61M 37/0015; A61K 9/0009; A61K 9/0019; A61K 9/0048; A61K 9/1652; A61K 31/728; A61P 27/02; A61M 2037/0023; A61M 2037/0061

(57) ABSTRACT

The present disclosure provides methods for administering formulations to the suprachoroidal space of the eye to achieve targeted drug delivery to a desired site of action within the eye. The methods include using a pushing formulation that includes a material such as a gel that expands due to physiochemical cues; or using an electric field to affect the movement of a formulation within the suprachoroidal space; or a combination of a pushing formulation with an electric field.

### CROSS-REFERENCE TO RELATED APPLICATIONS

This application is a continuation application of U.S. Utility application Ser. No. 18/802,027, filed Aug. 13, 2024, which is a continuation application of U.S. Utility application Ser. No. 16/609,583, now U.S. Pat. No. 12,090,294, filed Oct. 30, 2019, which is the National Stage of International Application No. PCT/US2018/030688, filed May 2, 2018, which claims priority to and the benefit of U.S. Provisional Patent Application Ser. No. 62/500,209, filed May 2, 2017; the disclosure of which is incorporated herein by reference in their entireties.

### BACKGROUND

The embodiments described herein relate generally to the field of drug delivery and more particularly to systems, devices, methods, and kits for targeted delivery of a substance into ocular tissues.

The anterior region of the eye refers to the front portion of the eye and includes structures in front of the vitreous humour such as the cornea, iris, ciliary body and lens. The posterior region of the eye refers to the back portion of the eye (i.e., the portion of the eye behind the lens), and includes the vitreous humor, retina, choroid, and optic nerve. The sclera is an opaque, fibrous, protective outer layer of the eye. The sclera includes connective tissue that maintains the shape of the eye by offering resistance to internal and external forces. The suprachoroidal space is the potential space or area between the sclera and choroid in the posterior region of the eye. Many inflammatory and proliferative diseases in the posterior region of the eye require long-term pharmaceutical treatment.

Although there are known methods of delivery of substances (e.g., drugs) into the posterior region of the eye, there is a need for improved devices and methods. It is often difficult to deliver effective doses of a drug to the back of the eye using conventional delivery methods such as topical application, intravitreal administration (IVT), or systemic administration. For example, topical applications, such as eye drops, are useful in treating conditions affecting the exterior surface of the eye or tissues at the front of the eye, however, eye drops are often not sufficiently conveyed to the back of the eye, as may be required for treatment of some retinal diseases such as macular degeneration, diabetic retinopathy, uveitis, and the like. Moreover, there is a short drug-eye contact time using eye drops, which can lead to more frequent applications of the drug. Other topical applications, such as ointments, allow a prolonged drug-eye contact time, thus requiring less frequent applications, but the application process increases the possibility of contamination since the drug is often applied via a person's hand. Furthermore, drugs that are administered via topical application are hindered from reaching the posterior region of the eye by components of the anterior region of the eye, as well as physiologic processes such as tears, blinking, drug metabolism, and drug binding.

Some known methods of treatment employ intravitreal (IVT) administration. IVT administration can include multiple injections due to the limited half-life of many compounds in the vitreous, potentially causing trauma and increase the risk of cataract, retinal detachment, hemorrhage and endophthalmitis.

The delivery of drugs to the posterior region of the eye through systemic administration is limited by the outer and inner blood-retinal barriers. Moreover, other limitations for systemic application of drugs include potentially reduced time of therapeutic effects and potency due to the dilution and degradation of the drug before reaching the target tissue. Thus, systemic administration usually requires an increase in the quantity of drugs necessary to achieve therapeutic concentrations at the target tissue, which increases the risk of adverse effects due to the accumulation of the drug in other tissues throughout the body.

The suprachoroidal space (SCS) of the eye has been studied as a location for drug delivery to posterior segments. The SCS can be reached via surgical means and/or cannulation. Although SCS delivery improves targeting to sites of drug action compared to other means such as topical, intravitreal, or systemic administration, it would be advantageous to achieve precisely controlled targeting to desired sites of drug action within the eye. This disclosure addresses this and other needs.

### SUMMARY

Drug delivery to the suprachoroidal space (SCS) using a microneedle targets drugs to the choroid and adjacent retina. Although SCS delivery improves targeting to sites of drug action, further control of drug distribution and targeting to specific tissues and areas in specific tissues of the eye will enable more precise drug targeting, and more effective treatment. The present disclosure provides methods, systems, devices, and compositions for targeted drug delivery to ocular tissues, comprising the use of a pushing formulation and/or iontophoresis. In one aspect, the present disclosure provides methods for delivery of a drug formulation to a target tissue in the eye of a subject, the method comprising administering to the suprachoroidal space (SCS) of the subject a first formulation and a second formulation, wherein the first formulation comprises an active agent and the second formulation is a pushing formulation. In some embodiments, the pushing formulation is a formulation of higher density and/or viscosity relative to the first formulation such that the administration of the pushing formulation following the first formulation results in pushing the first formulation toward the posterior segment of the eye.

In one aspect, the present disclosure provides methods for delivery of a drug formulation to a target tissue in the eye of a subject, the method comprising administering to the suprachoroidal space (SCS) of the subject a formulation comprising an active agent and further comprising applying iontophoresis to the eye of the subject to localize the active agent to a target tissue in the eye. In one aspect, the present disclosure provides methods for delivery of a drug formulation to a target tissue in the eye of a subject, the method comprising using a combination of a pushing formulation and iontophoresis.

In some embodiments, the first formulation and the second formulation are administered to the SCS using a microneedle. In some embodiments, the microneedle is a hollow microneedle. In some embodiments, the microneedle is between about 600 μm and about 1200 μm in length. In further embodiments, the microneedle is between about 900 and 1100 μm in length. In further embodiments, the microneedle is about 750 μm in length. In some embodiments, the microneedle is about 900 μm in length. In some embodiments, the microneedle is about 1100 μm in length. In some embodiments, the microneedle size is 28 gauge or smaller. For example, in some embodiments, the microneedle is a 30 gauge microneedle, a 31 gauge microneedle, a 32 gauge microneedle, or a 33 gauge microneedle.

In some embodiments, the first formulation and the second formulation are administered to the SCS using a device containing both the first and second formulation. In some embodiments, the first formulation is administered to the SCS prior to administration to the SCS of the pushing formulation. In some embodiments, the first formulation has a lower viscosity than the pushing formulation. In some embodiments, the first formulation has a lower density than the pushing formulation. In some embodiments, the pushing formulation comprises a gel. In some embodiments, the pushing formulation comprises hyaluronic acid (HA). In some embodiments, the pushing formulation comprises about 1% (w/v), about 2% (w/v), about 3% (w/v), about 4% (w/v), or about 5% (w/v) HA. In some embodiments, the pushing formulation comprises a salt. For example, in some embodiments, the pushing formulation is a high-salt formulation. For example, in some embodiments, the pushing formulation comprises 9% sodium chloride (NaCl). In certain embodiments, the pushing formulation comprises about 4% (w/v) HA and about 9% NaCl.

In some embodiments, the first formulation comprises an active agent useful in the treatment of an ocular disease or disorder. In some embodiments, the first formulation comprises particles, e.g., a suspension of particles. In certain embodiments, the particles are microparticles or nanoparticles. In some embodiments, the first formulation comprising the active agent further comprises HA. In some embodiments, the first formulation comprises about 0.1% (w/v) HA, about 0.5% (w/v) HA, about 1% (w/v) HA, about 2% (w/v) HA, or about 3% (w/v) HA. In certain embodiments, the first formulation comprises about 1% (w/v) HA. In some embodiments, the first formulation comprises about 1% (w/v) HA and the pushing formulation comprises about 4% (w/v) HA.

In some embodiments, the target tissue of the eye is selected from the group consisting of the posterior pole, optic nerve, choroid, retina, vitreous humor, macula, iris, or ciliary body. In some embodiments, the methods provided herein comprise the use of a pushing formulation that pushes the drug formulation further posteriorly in the posterior segment of the eye. In some embodiments, the methods provided herein result in localization of at least 10%, at least 20%, at least 30%, at least 40%, or at least 50% of the first formulation and/or the active agent therein to a location in the eye that is at least about 3 mm posterior to the limbus. In further embodiments, the methods provided herein result in localization of at least 10%, at least 20%, at least 30%, at least 40%, or at least 50% of the first formulation and/or the active agent therein to a location in the eye that is at least about 6 mm posterior to the limbus. In some embodiments, the methods provided herein result in localization of at least 10%, at least 20%, at least 30 %, or more of the first formulation and/or the active agent therein to a location in the eye that is at least 9 mm posterior to the limbus.

In one aspect, the present disclosure provides a device comprising a microneedle and a syringe or drug formulation reservoir, wherein the device comprises a first formulation and a second formulation, wherein the first formulation comprises an active agent and the second formulation is a pushing formulation. In some embodiments, the syringe is coupled to a microneedle. In some embodiments, the first formulation and the second formulation are substantially separate. For example, in some embodiments, the term “substantially separate” means there is little mixing or substantially no mixing of the first formulation and the second formulation. In some embodiments, there is mixing of the first and second formulations prior to, during, and/or after infusion of the formulations into the SCS, but the extent of the mixing does not prevent the pushing of the first formulation comprising the active agent further posteriorly in the posterior segment of the eye.

In some embodiments, the ocular disease or disorder is selected from the group consisting of uveitis, retinal vein occlusion, diabetic retinopathy, macular edema, diabetic macula edema, age-related macular degeneration, and glaucoma. In some embodiments, the disease or disorder amenable to treatment with the methods and compositions provided herein is selected from the group consisting of retinal vein occlusion, diabetic retinopathy, diabetic macula edema, and age-related macular degeneration, wherein the first formulation is injected to the posterior segment of the eye, and wherein the pushing formulation further pushes the first formulation posteriorly in the posterior segment of the eye. In some embodiments, the present disclosure provides methods wherein the active agent or drug in the first formulation flows or diffuses to the retina. In some embodiments, the disease or disorder amenable to treatment with the methods and compositions provided herein is glaucoma, and wherein the pushing formulation pushes the first formulation to the ciliary body.

In some embodiments, the present disclosure provides the use of a first formulation and a second formulation in a method of delivering a drug formulation to a target tissue in the eye of a subject, wherein the first formulation and second formulation are delivered to the suprachoroidal space (SCS) of the eye of the subject, wherein the first formulation comprises an active agent and the second formulation is a pushing formulation. In some embodiments, the present disclosure provides a first formulation and a second formulation for use in a method of treating a posterior ocular disease or disorder in a subject in need thereof, wherein the first formulation and second formulation are delivered to the suprachoroidal space (SCS) of the eye of the subject, wherein the first formulation comprises an active agent and the second formulation is a pushing formulation.

In some embodiments, the present disclosure provides the use of a first formulation and a second formulation in a method of delivering a drug formulation to a target tissue in the eye of a subject, wherein the first formulation and second formulation are delivered to the suprachoroidal space (SCS) of the eye of the subject, wherein the first formulation comprises a pushing formulation and the second formulation comprises an active agent. In some embodiments, the methods provide a means for pushing the active agent of the second formulation anteriorly. For example, in some embodiments, the methods provide a means for pushing an active agent toward the limbus of the eye. For example, in some embodiments, the methods provide a means for targeting an active agent to the ciliary body. In some embodiments, the present disclosure provides methods for treating a disease or disorder in a subject in need thereof, the method comprising administering to the SCS of the eye of the subject a first formulation that is a pushing formulation, and subsequently administering to the SCS of the eye of the subject a second formulation comprising a drug or active agent. In some embodiments, the disease or disorder is glaucoma.

In some embodiments, the methods provided herein involve iontophoresis, wherein the formulation comprising the active agent is administered to a region of the eye, e.g., the SCS, prior to the application of iontophoresis to the eye. In some embodiments, the formulation comprising the active agent is administered to the SCS concurrently with the application of iontophoresis to the eye. In some embodiments, the iontophoresis applied to the eye comprises applying a current with an absolute value of about 0.05 mA to about 0.8 mA. In some embodiments, the iontophoresis applied to the eye comprises applying a current with an absolute value of about 0.1 to about 0.7 mA. In some embodiments, the current has an absolute value of about 0.07 mA, about 0.08 mA, about 0.09 mA, about 0.1 mA, about 0.11 mA, about 0.12 mA, about 0.13 mA, about 0.14 mA, about 0.15 mA, about 0.16 mA, about 0.17 mA, about 0.18 mA, about 0.19 mA, about 0.2 mA, about 0.25 mA, about 0.3 mA, about 0.35 mA, about 0.4 mA, or about 0.5 mA. In some embodiments, a positive or negative current is used with an active agent and/or particle having an opposite polarity, for example, to move the active agent further posteriorly after injection. In some embodiments, the current with an absolute value provided herein is a positive current and is used with an active agent and/or particle having a negative charge. In some embodiments, the current with an absolute value provided herein is a negative current and is used with an active agent and/or particle having a positive charge. In other embodiments, a positive or negative current is used with an active agent and/or particle having the same polarity, for example to keep the active agent in the anterior part of the SCS (e.g., the ciliary body) after injection. For example, in some embodiments, the current with an absolute value provided herein is a negative current and is used with an active agent and/or particle having a negative charge; or the current with an absolute value provided herein is a positive current and is used with an active agent and/or particle having a positive charge

In some embodiments, the iontophoresis is applied to the eye for about 1 minute to about 10 minutes. In some embodiments, the iontophoresis is applied to the eye for about 3 minutes.

In one aspect, the present disclosure provides methods for delivery of a drug formulation to a target tissue in the eye of a subject, the method comprising (i) administering to SCS of the eye of the subject a first formulation and a second formulation, wherein the first formulation comprises an active agent and the second formulation is a pushing formulation; and (ii) applying iontophoresis to the eye of the subject to further localize the active agent to a target tissue in the eye. In some embodiments, the method results in localization of the active agent further posteriorly in the posterior segment of the eye, such as precise localization to desired tissues within the eye or specific areas on desired tissues within the eye. In some embodiments, the method results in localization of at least about 30% of the first formulation to a location in the eye that is at least about 6 mm posterior to the limbus. In further embodiments, method results in localization of at least about 50% of the first formulation to a location in the eye that is at least about 6 mm posterior to the limbus. In some embodiments, the iontophoresis is applied to the eye prior to the administration of the second formulation. In other embodiments, the iontophoresis is applied to the eye during and/or after administration of the second formulation. In one aspect, the present disclosure provides a method comprising (i) administering to the SCS of the eye of the subject a first formulation comprising an active agent; (ii) applying iontophoresis to the eye of the subject to localize the active agent to a target tissue in the eye; and (iii) administering to the SCS of the eye of the subject a second formulation, wherein the second formulation is a pushing formulation and wherein the second formulation further localizes the active agent to a target tissue in the eye.

### BRIEF DESCRIPTION OF THE DRAWINGS

FIG. 1 provides schematic views of a cross-section of a human eye and an SCS injection thereof. Schematic diagrams of the eye following pushing hydrogel, iontophoresis, and combination pushing with iontophoresis embodiments of the present disclosure are also provided.

FIG. 2A is a schematic diagram of a microneedle with a syringe containing a particle formulation and a hydrogel formulation. FIG. 2B is a photographic image of a microneedle with a syringe containing a particle formulation and a hydrogel formulation.

FIG. 3 is a schematic diagram of the SCS injection of a hydrogel pushing formulation and its effects within the suprachoroidal space. Before injection (box (i)), the SCS is the potential space between the sclera (S) and the choroid (Ch). The retina (R) is located between the choroid and the vitreous (V). Box (ii) shows the suprachoroidal space after SCS injection of a particle formulation followed by a hydrogel pushing formulation. Box (iii) shows the suprachoroidal space after the hydrogel pushing formulation has caused spreading through the SCS, pushing the particle formulation toward the posterior segment of the eye.

FIG. 4A shows the particle distribution (% of particles located at the indicated positions) when the pushing formulation is held at 4% HA and the drug particle formulation is tested at 0%, 1%, 2%, or 4% HA. The averaged particle transfer (APT) for each group is also shown at the top of the graph for each group. FIG. 4B shows the particle distribution (% of particles located at the indicated positions) when the drug particle formulation is held at 1% HA and the pushing formulation is tested at 2%, 4%, or 8% HA. The APT is also shown at the top of the graph for each group. Averaged particle transfer (APT) (mm)=(1.5 mm×the particle distribution % of 0-3 mm area)+(4.5 mm×the particle distribution % of 3-6 mm area)+(7.5 mm×the particle distribution % of 6-9 mm area)+(10.5 mm×the particle distribution % of 9-mm area). R is the reference value. ▾, ▾▾Significance (One-way ANOVA) between the APT of the reference (R) and the APT of the other formulations. *Significance (One-way ANOVA) between the 0-3 mm distribution of the reference (R) and the other formulations. The average±standard deviation based on 3 replicate samples (avg±SD, n=3) is shown in both FIG. 4A and FIG. 4B. FIG. 4C shows the digital (i) and fluorescent (ii) images after dissection, when dissection was performed right after SCS injection.

FIG. 5 shows the particle distribution (%) at each region in the eye following SCS injection of the 1%/4% formulation and incubation and no incubation ((i), 0 hr), for 6 hours at 4° C. (ii), for 6 hours at room temperature (iii), or for 6 hours at 37° C. (iv). The averaged particle transfer (APT) is also shown in mm at the top of the bars in the graph for each group. R is reference value. ▴Significance (One-way ANOVA) between the APT of the reference (R) and the APT of the other formulations. *,**Significance (One-way ANOVA) between the 9-mm distribution of the reference (R) and other formulations. The graphs show the average±standard deviation based on 3 replicate samples (avg±SD, n=3).

FIG. 6 shows the bright field (top row) and fluorescent (bottom row) images of ex vivo rabbit eyes after SCS injection of the 1%/4% formulation and no incubation (1^{st} column), for 6 hours at 4° C. (2^{nd} column), for 6 hours at room temperature (RT; 3^{rd} column), or for 6 hours at 37° C. (4^{th} column).

FIG. 7A shows a digital image of a dissected eye right after the injection of the 1%/4% formulation. FIG. 7B shows a digital image of a dissected eye after a 6 hour incubation at 37° C. following the injection.

FIG. 8A shows the particle distribution (bar graph) and bright field and fluorescent images immediately following SCS injection of 50 μL of red fluorescent particles in HBSS buffer (no HA). APT was 4.2±0.3 mm. FIG. 8B shows the particle distribution (bar graph) and bright field and fluorescent images immediately following SCS injection of 20 μL of particle in 1% HA. APT was 3.6±0.4 mm. FIG. 8C shows the particle distribution (bar graph) and bright field and fluorescent images following SCS injection of 20 μL of particle in 1% HA, after a 6 hour incubation at 37° C. APT was 3.7±0.5 mm. R is reference value. No Significance (One-way ANOVA) between the APT of the reference (R) and the APT of the formulations. Graphs (a) present average±standard deviation based on 3 replicate samples (avg±SD, n=3).

FIG. 9A is a schematic showing the 1% HA drug particle formulation and silicone oil in a microneedle. FIG. 9B shows the percent particle distribution at the indicated positions immediately after the injection (0 hr), or after a 6 hour incubation at 37° C. following the injection, using the silicone oil as the pushing formulation. The APT in the 0 hr group was 3.6±0.7 mm. The APT in the 6 hr incubation group was 4.7±0.6 mm. FIG. 9C shows the bright field and fluorescent images for the two groups. R is reference value. No Significance (One-way ANOVA) between the APT of the reference (R) and the APT of the formulations. Graphs (a) present average±standard deviation based on 3 replicate samples (avg±SD, n=3).

FIG. 10 shows the particle distribution of red fluorescent particles immediately after ex vivo injection (i), after incubation at 37° C. for 6 hours following the ex vivo injection of the HA hydrogel (without added salt) (ii), after incubation at 37° C. for 6 hours following ex vivo injection of the HA hydrogel with 9% sodium chloride added (iii; “HS_HA”), two days after in vivo injection into rabbit eyes using the high-salt (9% sodium chloride) HA formulation. The APT is shown above the bars for each group. R is reference value. ▴,▴▴Significance (One-way ANOVA) between the APT of the reference (R) and the APT of the formulations. *,**Significance (One-way ANOVA) between the 9-mm distribution of the reference (R) and other formulations. Graphs (a) present average±standard deviation based on 3 replicate samples (aver±SD, n=3).

FIG. 11A shows the bright field and fluorescent images for the high-salt HA group (6 hour incubation at 37° C. FIG. 11B shows the bright field and fluorescent images for the in vivo injection of high-salt HA pushing formulation.

FIG. 12 is a schematic showing the concentration of drug particles at the back of the eye when the high-salt HA pushing formulation was used.

FIG. 13A is a schematic providing the locations at which SCS thickness was measured. FIG. 13B shows the SCS thickness (mm) at each measured location and at 0 hr, 4 hr, or 2 days following injection of the HA hydrogel. FIG. 13C shows the SCS thickness (mm) at each measured location and at 0 hr, 4 hr, or 2 days following injection of the high-salt HA hydrogel.

FIG. 14 is a schematic diagram of the SCS injection with iontophoresis application and its effects within the suprachoroidal space. Before injection (box (i)), the SCS is the potential space between the sclera (S) and the choroid (Ch). The retina (R) is located between the choroid and the vitreous (V). Box (ii) shows the suprachoroidal space after SCS injection of a particle formulation and before application of iontophoresis. Box (iii) shows the suprachoroidal space after the application of iontophoresis, wherein the applied current drives particle movement within the SCS toward the posterior segment of the eye.

FIG. 15 provides photo images of exemplary materials for use in the iontophoretic microneedle system for injection, for example for injection into a rabbit eye ex vivo. The left panel and top right panel show an injection syringe with microneedle, wherein the injection syringe contains an electrode mounted on the end of the plunger and a Pd wire. The other electrode is attached at the end of the optic nerve as shown in the left panel image. The bottom right panel shows a microneedle holder with an electrolyte gel added to facilitate electrical connectivity between the microneedle device and the ocular tissue, and also to reduce the heat generated from the microneedle during iontophoresis application.

FIGS. 16A and 16B provide the fluorescence micrographs and % particle distribution, respectively, following 50 μL injection of the red fluorescent particle formulation, without (i) or with (ii) iontophoresis of 0.14 mA for 3 min following the injection. FIGS. 16C and 16D provide the fluorescence micrographs and % particle distribution, respectively, following 100 μL injection of the red fluorescent particle formulation, without (i) or with (ii) iontophoresis of 0.14 mA for 3 min following the injection. FIGS. 16E and 16F provide the fluorescence micrographs and % particle distribution, respectively, following 200 μL injection of the red fluorescent particle formulation, without (i) or with (ii) iontophoresis at 0.14 mA for 3 min following the injection. The fluorescence micrographs (FIGS. 16A, 16C, 16E) show representative flat mounts of the eye after dissection eye with radial cuts from the posterior pole to the limbus. The arrows point to sites of injection into the SCS. The average particle transfer (APT) for each group is provided in FIG. 16G. Total distance of the particle transfer was calculated based on the particle distribution profile, averaged particle transfer (APT). Averaged particle transfer (APT) (mm)=(1.5 mm×the particle distribution % of 0-3 mm area) +(4.5 mm×the particle distribution % of 3-6 mm area)+(7.5 mm×the particle distribution % of 6-9 mm area)+(10.5 mm×the particle distribution % of 9-mm area). *,**Significance (One-way ANOVA, p<0.00024, 0.00070, and 0.03026, respectively) between without iontophoresis and with iontophoresis samples. Two-way ANOVA: 1) volume (p<0.000053), 2) current (p<0.00000043), and interaction of v and c (p<0.7547790). Graphs (FIGS. 16B, 16D, 16F, 16G) present average±standard deviation based on 3 replicate samples (avg±SD, n=3).

FIG. 17A provides fluorescence micrographs following SCS injection of red fluorescent particles and no iontophoresis (i), or iontophoresis at 0.14 mA (ii) or 0.7 mA (iii). The fluorescence micrographs show the representative flat mounts of the eyes after dissecting eyes with radial cuts from the posterior pole to the limbus. The arrows point to sites of injection into the SCS. FIG. 17B shows the particle distribution to the 0-3 mm, 3-6 mm, 6-0 mm, or 9-mm area in the SCS. *,**Significance (One-way ANOVA, p<0.00127, 0.04985, and 0.00707, respectively) between (i) and (ii), (ii) and (iii), and (i) and (iii) of the 9-mm distributions. FIG. 17C shows the APT (mm) for each group. *Significance (One-way ANOVA, p<0.03026, 0.01636, and 0.01264, respectively) between (i) and (ii), (ii) and (iii), and (i) and (iii) of the APT. In FIGS. 17B and 17C, the graphs present average±standard deviation based on 3 replicate samples (avg±SD, n=3).

FIG. 18A provides fluorescence micrographs following SCS injection of red fluorescent particles and iontophoresis application time of 1.5 min (i), 3 min (ii), or 5 min (iii). The fluorescence micrographs show the representative flat mounts of the eyes after dissecting eyes with radial cuts from the posterior pole to the limbus. The arrows point to sites of injection into the SCS. FIG. 18B shows the particle distribution to the 0-3 mm, 3-6 mm, 6-0 mm, or 9-mm area in the SCS. **Significance (One-way ANOVA, p<0.00073 and 0.00791) between (i) and (ii), and (ii) and (iii) of the 9-mm distributions. FIG. 18C shows the APT (mm) for each group. **Significance (One-way ANOVA, p<0.01680 and 0.01932) between (i) and (ii), and (i) and (iii) of the APT. In FIGS. 18B and 18C, the graphs present average±standard deviation based on 3 replicate samples (avg±SD, n=3)

FIG. 19A is a line graph showing the thickness of the SCS over time with different injection volumes (50, 100, or 200 μL). The thickness was measured at 1 minute intervals from 0 min to 10 min. FIG. 19B shows ultrasound images of the rabbit eye ex vivo before injection (0 min, (i)), 1 minute after injection (ii), or 5 minutes after injection (iii) with an injection volume of 50 μL. FIG. 19C shows ultrasound images of the rabbit eye ex vivo before injection (0 min, (i)), 1 minute after injection (ii), or 5 minutes after injection (iii) with an injection volume of 100 μL. FIG. 19D shows ultrasound images of the rabbit eye ex vivo before injection (0 min, (i)), 1 minute after injection (ii), or 5 minutes after injection (iii) with an injection volume of 200 μL.

FIG. 20A shows ultrasound images of the rabbit eye ex vivo after SCS injection with 50 μL at 0 min (i), 1 min (ii), 2 min (iii), 3 min (iv), 4 min (v), or 5 min (vi). FIG. 20B shows ultrasound images of the rabbit eye ex vivo after SCS injection with 100 μL at 0 min (i), 1 min (ii), 2 min (iii), 3 min (iv), 4 min (v), or 5 min (vi). FIG. 20C shows ultrasound images of the rabbit eye ex vivo after SCS injection with 200 μL at 0 min (i), 1 min (ii), 2 min (iii), 3 min (iv), 4 min (v), or 5 min (vi).

FIG. 21A is a schematic diagram depicting the reversed current study. FIG. 21B shows the fluorescence micrographs providing the distributions of the red fluorescent particles in the SCS after no iontophoresis (i) or after the iontophoretic direction was reversed (−0.07 mA; (ii)). The fluorescence micrographs show representative flat mounts of the eye after dissection eye with radial cuts from the posterior pole to the limbus. The arrows point to sites of injection into the SCS. FIG. 21C provides the particle distribution %, and FIG. 21D shows the APT. **Significance (One-way ANOVA, p<0.00131) between (i) 0 mA and (ii) −0.07 mA of the APT. For 21C and 21D, the graphs present average±standard deviation based on 3 replicate samples (avg±SD, n=3).

FIG. 22 is a photograph image of an exemplary in vivo delivery using the iontophoretic ocular drug delivery system. The microneedle embedding a Ag/AgCl electrode was injected to the SCS from supranasal location of the eye. The other Ag/AgCl electrode was attached on the ear of the rabbit.

FIGS. 23A-23C provide the results of in vivo injection of 100 μL red fluorescent particles using iontophoresis. The iontophoresis condition was 0.14 mA for 3 minutes. FIG. 23A provides fluorescent micrographs showing the distribution of the red-fluorescent particles in the SCS after injection at time 0 (i), 1 hour after injection (ii), and 1 week after injection (iii). The micrographs show representative flat mounts of the eyes after dissecting eyes with radial cuts from the posterior pole to the limbus. The arrows point to sites of injection into the SCS. FIG. 23B shows the particle distribution percent after injection at each time point. *,**Significance (One-way ANOVA, p<0.0064 and 0.0487) between (i) and (ii), and (ii) and (iii) of the 9-mm distributions. FIG. 23C shows the APT at each time point. *,**Significance (One-way ANOVA, p<0.00401 and 0.04367) between (i) and (ii), and (i) and (iii) of the APT. For FIGS. 23B and 23C, graphs present average±standard deviation based on 3 replicate samples (avg±SD, n=3).

FIG. 24A shows the red particles by fluorescent micrograph 1 hour after in vivo injection of 100 μL red fluorescent particles and iontophoresis using a current of 0.14 mA for 3 minutes (as shown in FIG. 23A(ii). FIG. 24B is a schematic diagram showing the particle distribution to the back of the eye, if the eye were reconstructed.

FIG. 25A shows a histological image of an untreated eye. FIG. 25B shows three images of eyes 1 day after in vivo injection and iontophoresis application. One eye showed no symptoms (i). One eye showed choroid inflammation (ii). One eye showed choroid/retinal damage (iii). FIG. 25C shows an eye 1 week after in vivo injection and iontophoresis application. Image (ii) is a magnification of image (i). No symptoms were observed in the eyes 1 week after the injection and iontophoresis application.

FIG. 26 provides a fluorescent image and a schematic depiction of one embodiment of the combination of iontophoresis with pushing hydrogel injection for delivery of formulations to the back of the eye.

FIG. 27 is an additional schematic depiction of one embodiment of the combination of iontophoresis with pushing hydrogel injection for delivery of formulations to the back of the eye.

FIG. 28A shows the results of different current conditions for injection of 50 μL drug particle formulation (without pushing formulation). Current conditions of 0.15 mA, 0.3 mA, and 0.5 mA were tested. FIG. 28B shows the results of different HA formulations for the pushing formulation (tested in the absence of iontophoresis). 1% HA, 2% HA, 3% HA, and 4% HA formulations were tested.

FIG. 29A shows the ex vivo setup for the combined iontophoresis and hydrogel pushing study. FIG. 29B shows the in vivo setup for the combined iontophoresis and hydrogel pushing study.

FIG. 30 shows the ex vivo results of a control injection (particles only, (a); particles are preferentially distributed in the anterior area); 4% hydrogel pushing after particle injection, without iontophoresis (b); iontophoresis only after particle injection (no hydrogel injection; (c)); the combination of iontophoresis and pushing formulation administration (iontophoresis was applied first, followed by hydrogel injection; (d)); or the combination of iontophoresis and pushing formulation administration and an incubation for 6 h at 37° C. to induce hydrogel swelling (e).

FIG. 31 shows the results of a comparison of the order of iontophoresis and gel pushing. The black squares show the results of application of iontophoresis prior to hydrogel injection. The white squares show the results of iontophoresis application after hydrogel injection.

FIG. 32 shows the results of the in vivo combination study. (a) is the control (50 μL particles only). (b) is iontophoresis only. (c) is iontophoresis+gel administration right after injection. (d) is iontophoresis+gel administration 1 week after the injection. (e) is iontophoresis+gel administration 2 weeks after the injection.

FIG. 33 shows the intraocular pressure (IOP) after the combination iontophoresis+hydrogel injection in three animals (N1, N2, and N3). IOP was reduced to around −4 mmHg after treatment. In N3, the IOP of the injected eye was reduced after the injection, but the IOP in the other eye (dashed line) was normal. In each animal, IOP recovered to the normal range around 9˜10 days after the injection.

FIG. 34 provides histology of a control (untreated) eye.

FIG. 35 provides histology of a treated eye (iontophoresis+hydrogel injection) 2 days after the injection. Although there was a slight inflammatory response, generally the retina and choroid, and other areas did not show significant damage.

FIG. 36 provides histology of a treated eye (iontophoresis+hydrogel injection) 2 weeks after the injection. Generally the retina, choroid, and other areas did not show significant damage.

### DETAILED DESCRIPTION

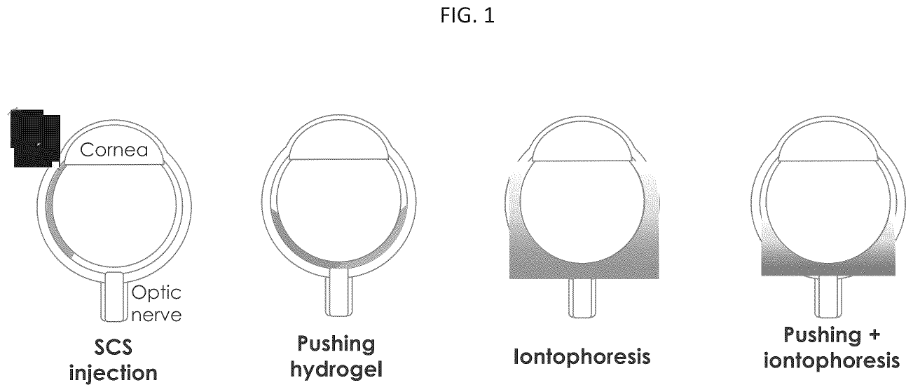

Devices, methods, systems, compositions, and kits for ocular drug delivery are described herein. FIG. 1 shows representative schematic diagrams of the pushing hydrogel, iontophoresis, and combined pushing with iontophoresis methods provided herein. In one aspect, the present disclosure provides methods for better drug delivery to the target tissue or site of action of drugs in the eye of a subject, via methods including injection into the suprachoroidal space (SCS) of the eye of the subject. For example, in some embodiments, the present disclosure provides systems, methods, and compositions for targeted delivery to one or more tissues such as the ciliary body, retina, choroid, and/or macula. In some embodiments, the systems, methods, and compositions involve injection of one or more formulations into the SCS of the eye, and control the delivery to the particular target tissues within the eye. For many indications (e.g., glaucoma, uveitis, macular degeneration), the sites of action can be at various locations around the eye that can be accessed via the SCS. Accordingly, targeted drug delivery within the SCS by accurate control of the drug localization is of interest to reduce potential risks of side-effects, such as those side effects caused by drugs spread off-target or widely spread adjacent to target tissues. In addition, targeted delivery within the SCS by accurate control of the drug localization increases drug concentration and efficacy at the site(s) of action. In one aspect, the present disclosure provides two novel targeted ocular drug delivery systems. One is driven by “hydrogel pushing” and one is driven by iontophoresis. In some embodiments, a combination of the hydrogel pushing and iontophoresis methods is provided. The methods provided herein are useful in treatment of diverse ocular diseases.

As used herein, “ocular tissue” and “eye” include both the anterior segment of the eye (i.e., the portion of the eye in front of the lens) and the posterior segment of the eye (i.e., the portion of the eye behind the lens).

The term “suprachoroidal space,” is used interchangeably with suprachoroidal, SCS, suprachoroid and suprachoroidia, and describes the potential space in the region of the eye disposed between the sclera and choroid. This region primarily is composed of closely packed layers of long pigmented processes derived from each of the two adjacent tissues; however, a space can develop in this region as a result of fluid or other material buildup in the suprachoroidal space and the adjacent tissues. The “supraciliary space,” as used herein, is encompassed by the SCS and refers to the most anterior portion of the SCS adjacent to the ciliary body, trabecular meshwork and limbus. Those skilled in the art will appreciate that the suprachoroidal space frequently is expanded by fluid buildup because of some disease state in the eye or as a result of some trauma or surgical intervention. In the present description, however, the fluid buildup is intentionally created by infusion of a drug formulation into the suprachoroid to create the suprachoroidal space (which is filled with drug formulation). Not wishing to be bound by theory, it is believed that the SCS region serves as a pathway for uveoscleral outflow (i.e., a natural process of the eye moving fluid from one region of the eye to the other through) and becomes a real space in instances of choroidal detachment from the sclera.

As used herein, “non-surgical” ocular drug delivery devices and methods refer to methods and devices for drug delivery that do not require general anesthesia and/or retrobulbar anesthesia (also referred to as a retrobulbar block). Alternatively or additionally, a “non-surgical” ocular drug delivery method is performed with an instrument having a diameter of 28 gauge or smaller. Alternatively or additionally, “non-surgical” ocular drug delivery methods do not require a guidance mechanism that is typically required for ocular drug delivery via a shunt or cannula. As used herein, “surgical” ocular drug delivery includes insertion of devices or administration of drugs by surgical means, for example, via incision to expose and provide access to regions of the eye including the posterior region, and/or via insertion of a stent, shunt, or cannula. In certain aspects, the non-surgical methods for delivery to the SCS provided herein are achieved by injecting a drug or drug formulation to the SCS using a microneedle.

In some embodiments, features of the devices, formulations, and methods are provided in International Patent Application Publication Nos. WO2014/074823 (Application No. PCT/US2013/069156), WO2015/195842 (Application No. PCT/US2015/036299), and/or WO2017/120601 (Application No. PCT/US2017/012757), each of which is hereby incorporated by reference in its entirety for all purposes.

In some embodiments, the present disclosure provides systems, methods, and compositions wherein a drug formulation (e.g., a formulation containing an active agent) may be delivered to a particular action site depending on the ocular disease for which the subject is being treated. Exemplary target sites, associated indications, and active agents or drug formulations for treatment of the indication are provided below. The person of ordinary skill in the art will appreciate that other target sites, indications, and active agents/drug formulations are encompassed by the invention. For example, the present disclosure provides compositions and methods for delivery of active agents and drug formulations to the optic nerve. Other target sites, indications, and active agents include, without limitation:
Conjunctiva: for treatment of conjunctivitis (e.g., antibiotic injection)
Cornea: for treatment of infectious keratitis (antimicrobial agent), non-infectious keratitis (steroid agent), corneal ulcer (antifungal/antiviral agent)
Sclera: for treatment of Scleritis (non-steroidal anti-inflammatory agent or steroid agent)
Uvea: for treatment of Uveitis (Iris, ciliary body, choroid), anterior/intermediate/posterior/panuveitis (steroid agent and immunosuppressant)
Ciliary body: for treatment Glaucoma (prostaglandin agent)
Retina: for treatment of retinal vein occlusion, diabetic retinopathy, diabetic macula edema, and age-related macular degeneration (anti-VEGF and steroid agent)

Ocular diseases and disorders amenable for treatment by the methods, systems, compositions, kits, devices, and drug formulations described herein can include, but are not limited to, uveitis (e.g., infectious uveitis, non-infectious uveitis, chronic uveitis, and/or acute uveitis), macular edema, diabetic macular edema (DME), macular edema associated with uveitis (encompassing macular edema associated with infectious uveitis and macular edema associated with non-infectious uveitis), macular edema following retinal vein occlusion (RVO), and macular edema associated with RVO. In one embodiment, the posterior ocular disorder is an ocular inflammatory condition such as uveitis, scleritis, glaucoma, ocular sarcoidosis, optic neuritis, macular edema, diabetic retinopathy, macular degeneration, a corneal ulcer, an autoimmune disorder, ophthalmic manifestations of AIDS, optic nerve degeneration, geographic atrophy, choroidal disease, retinitis, ocular neovascularization, polypoidal choroidal vasculopathy, choroidal sclerosis, central sirrus choroidopathy, a multifocal choroidopathy or a choroidal dystrophy (e.g., central gyrate choroidal dystrophy, serpiginous choroidal dystrophy, total central choroidal atrophy The condition in one embodiment is acute. In another embodiment, the condition is chronic.

Disorders caused by pathogenic organisms that can lead to infectious uveitis, macular edema associated with infectious uveitis, or other ocular disorders amenable to the treatment methods, systems, and compositions provided herein include, but are not limited to, toxoplasmosis, toxocariasis, histoplasmosis, herpes simplex or herpes zoster infection, tuberculosis, syphilis, sarcoidosis, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, or iridocyclitis.

The term “subject” may be used interchangeably herein with “patient” and the like. In some embodiments, the subject is any animal, including any mammal. In some embodiments, the subject is a human. In some embodiments, the subject is a human subject in need of treatment of an ocular disease or disorder.

### Delivery Materials

Drugs, molecules, particles, hydrogels, polymers, diagnostics, therapeutics, cosmetics/cosmeceuticals, prostheses/implants and other agents (materials) can be delivered into the suprachoroidal space of the eye. The material to be delivered could carry a positive charge, negative charge or no charge, and if zwitterionic, a net positive charge, net negative charge or no net charge. Thus, in some embodiments, the active agent or drug formulation useful in the compositions, methods, and systems provided herein comprises a charged moiety. The charged moiety may be created using any known technique, and generally may include a moiety that is altered to have a different charge (i.e., neutral to positive, neutral to negative, negative to positive, etc.), an increased charge, or decreased charge. In some embodiments, the charged moiety is a moiety that is positively charged under assay or physiological conditions, which are typically neutral or slightly acidic (pH about 5 to about 7). The positively charged moiety may include an amine. In another embodiment, the charged moiety is a moiety that is negatively charged under assay or physiological conditions. The negatively charged moiety may include a carboxylate, a phosphate, or a combination thereof. In some embodiments, the “first formulation” or “active agent formulation” and the like comprises the charged moieties, drugs, molecules, particles, hydrogels, polymers, diagnostics, therapeutics, cosmetics/cosmeceuticals, prostheses/implants, other agents provided herein.

As provided throughout, in one embodiment, the methods described herein are carried out with a hollow or solid microneedle, for example, a rigid microneedle. As used herein, the term “microneedle” refers to a conduit body having a base, a shaft, and a tip end suitable for insertion into the sclera and other ocular tissue and has dimensions suitable for minimally invasive insertion and drug formulation infusion as described herein. That is, the microneedle has a length or effective length that does not exceed about 2000 microns and a diameter that does not exceed about 600 microns. Both the “length” and “effective length” of the microneedle encompass the length of the shaft of the microneedle and the bevel height of the microneedle. In some embodiments, the microneedle used to carry out the methods described herein comprises one of the devices disclosed in International Patent Application Publication No. WO2014/179698 (Application No. PCT/US2014/036590), filed May 2, 2014 and entitled “Apparatus and Method for Ocular Injection,” incorporated by reference herein in its entirety for all purposes. In some embodiments, the microneedle used to carry out the methods described herein comprises one of the devices disclosed in International Patent Application Publication No. WO2014/036009 (Application No. PCT/US2013/056863), filed Aug. 27, 2013 and entitled “Apparatus and Method for Drug Delivery Using Microneedles,” incorporated by reference herein in its entirety for all purposes.

In some embodiments, features of the devices, formulations, and methods are provided in International Patent Application Publication Nos. WO2014/074823 (Application No. PCT/US2013/069156), WO2015/195842 (Application No. PCT/US2015/036299), and/or WO2017/120601 (Application No. PCT/US2017/012757), each of which is hereby incorporated by reference in its entirety for all purposes. In one embodiment, the device used to carry out one of the methods described herein comprises the device described in U.S. Design Patent Application Ser. No. 29/506,275 entitled, “Medical Injector for Ocular Injection,” filed Oct. 14, 2014, the disclosure of which is incorporated herein by reference in its entirety for all purposes. In one embodiment, the device used to carry out one of the methods described herein comprises the device described in U.S. Patent Publication No. 2015/0051581 or U.S. Patent Publication No. 2017/0095339, which are each incorporated herein by reference in their entireties for all purposes.

In some embodiments, the words “proximal” and “distal” when used herein in relation to an operator of the device refer to the direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of a microneedle described herein first inserted inside the patient's body would be the distal end, while the opposite end of the microneedle (e.g., the end of the medical device being manipulated by the operator) would be the proximal end of the microneedle. In other embodiments, the words “proximal” and “distal” when used herein in relation to a device itself may refer to the direction closer to or away from, respectively, the indicated end of the device. Thus, in some embodiments, a microneedle coupled to a syringe containing an active agent formulation and a pushing formulation may comprise an active agent formulation that is proximal to the needle end and a pushing formulation distal from the needle end, such that upon insertion of the microneedle and infusion into the SCS of the eye, the active agent formulation enters the SCS prior to the entry into the SCS of the pushing formulation.

As used herein, the terms “about” and “approximately” generally mean plus or minus 10% of the value stated. For example, about 0.5 would include 0.45 and 0.55, about 10 would include 9 to 11, about 1000 would include 900 to 1100.

As used herein, the term “hollow” (e.g., in relation to a hollow microneedle) includes a single, straight bore through the center of the microneedle, as well as multiple bores, bores that follow complex paths through the microneedles, multiple entry and exit points from the bore(s), and intersecting or networks of bores. That is, a hollow microneedle has a structure that includes one or more continuous pathways from the base of the microneedle to an exit point (opening) in the shaft and/or tip portion of the microneedle distal to the base.

In one embodiment, the microneedle has an effective length of about 50 μm to about 2000 μm. In another particular embodiment, the microneedle has an effective length of from about 150 μm to about 1500 μm, or from about 300 μm to about 1250 μm, or from about 500 μm to about 1250 μm, or from about 500 μm to about 1500 μm, or from about 600 μm to about 1000 μm, or from about 700 μm to about 1000 μm. In one embodiment, the effective length of the microneedle is about 600 μm, or about 700 μm, or about 800 μm or about 1000 μm. In various embodiments, the proximal portion of the microneedle has a maximum width or cross-sectional dimension of from about 50 μm to 600 μm, or from about 50 μm to about 400 μm, or from about 50 μm to about 500 μm, or from about 100 μm to about 400 μm, or from about 200 μm to about 600 μm, or from about 100 μm to about 250 μm, with an aperture diameter of about 5 μm to about 400 μm. In a particular embodiment, the proximal portion of the microneedle has a maximum width or cross-sectional dimension of about 600 μm. Those skilled in the art will appreciate, however, that in embodiments in which the tip of the microneedle is beveled that the aperture diameter may be greater than the outer diameter of the proximal portion of the microneedle. The microneedle may be fabricated to have an aspect ratio (width:length) of about 1:1.5 to about 1:10. In one embodiment, the aspect ratio of the microneedle is about 1:3 to about 1:5. In another embodiment, the aspect ratio of the microneedle is about 1:4 to about 1:10.

The microneedle can have a straight or tapered shaft. In one embodiment, the diameter of the microneedle is greatest at the base end of the microneedle and tapers to a point at the end distal the base. The microneedle can also be fabricated to have a shaft that includes both a straight (i.e., untapered) portion and a tapered (e.g., beveled) portion. In various embodiments the microneedle has a bevel angle of about 5 degrees to about 30 degrees, of about 5 degrees to about 25 degrees, about 5 degrees to about 20 degrees, about 10 degrees to about 20 degrees, and about 10 degrees to about 30 degrees. The microneedles can be formed with shafts that have a circular cross-section in the perpendicular, or the cross-section can be non-circular. The tip portion of the microneedles can have a variety of configurations. The tip of the microneedle can be symmetrical or asymmetrical about the longitudinal axis of the shaft. The tips may be beveled, tapered, squared-off, or rounded. In various embodiments, the microneedle has a bevel height from about 50 μm to 500 μm, about 100 μm to about 500 μm, about 100 μm to about 400 μm, about 200 μm to about 400 μm, and about 300 μm to about 500 μm. In particular embodiments, the microneedle may be designed such that the tip portion of the microneedle is substantially the only portion of the microneedle inserted into the ocular tissue (i.e., the tip portion is greater than 75% of the total length of the microneedle, greater than 85% of the total length of the microneedle, or greater than about 95% of the total length of the microneedle). In other particular embodiments, the microneedle may be designed such that the tip portion is only a portion of the microneedle that is inserted into the ocular tissue and generally has a length that is less than about 75% of the total length of the microneedle, less than about 50% of the total length of the microneedle, or less than about 25% of the total length of the microneedle. For example, in one embodiment the microneedle has a total effective length between 500 μm and 1500 μm, wherein the tip portion has a length that is less than about 400 μm, less than about 300 μm, or less than about 200 μm.

In one embodiment, the height of the bevel is about 100 μm to about 500 μm. In another embodiment, the height of the bevel is about 500 μm or less, about 450 μm or less, about 400 μm or less or about 350 μm or less. In another embodiment, the height of the bevel is from about 200 μm to about 500 μm, or from about 100 μm to about 700 μm, or from about 200 μm to about 700 μm. In still other embodiments, the height of the bevel is from about 500 μm to about 900 μm, or from about 500 μm to about 800 μm, or from about 500 μm to about 700 μm. In this manner, the arrangement of the bevel can be such that the distal edge is sufficiently sharp such as to pierce a target tissue and penetrate into the vitreous without (i) substantially causing the target tissue to elastically deform or (ii) damaging internal structures of the eye, e.g., the lens or retina.

In one embodiment, the microneedle extends from a base. The base may be integral with or separate from and coupled (e.g., slidably coupled or fixedly coupled) to the microneedle. The base may be rigid or flexible. The base may be substantially planar or it may be curved, for example, in the shape of the ocular tissue surface at the site of injection or, for example, curved away from the ocular surface (e.g., convex) so as to minimize contact between the base and the ocular tissue. In some embodiments, desirably, the base is shaped to provide minimal contact with the surface of the eye at the point of insertion. For example, in such embodiments, the base may extend only a minimal distance from the microneedle shaft substantially perpendicular. In another embodiment, the base may be shaped so as to elevate the ocular tissue towards the microneedle so as to counteract the deflection of the ocular tissue and facilitate insertion of the microneedle into the ocular tissue (e.g., the base may extend from the microneedle toward the tip portion of the microneedle so as to “pinch” the ocular tissue). Some such embodiments may be based, at least in part, on the devices described in U.S. Pat. No. 6,743,211, incorporated herein by reference.

Microneedles may be manufactured using techniques described, for example, in U.S. Patent Application Publication No. 2006/0086689, U.S. Patent Application Publication No. 2006/0084942, U.S. Patent Application Publication No. 2005/0209565, U.S. Patent Application Publication No. 2002/0082543, U.S. Pat. Nos. 6,334,856, 6,611,707, 6,743,211 and PCT/US2014/36590, filed May 2, 2014, all of which are incorporated herein by reference in their entireties for all purposes.

In some embodiments, the microneedle comprises a fluid reservoir for containing the drug formulation and/or the hydrogel pushing formulation. In some embodiments, the fluid reservoir is in operable communication with the bore of the microneedle at a location distal to the tip end of the microneedle. The fluid reservoir may be integral with the microneedle, integral with the elongated body, or separate from both the microneedle and elongated body. In some embodiments, the microneedle is coupled to, attached to, or configured to allow attachment to a syringe comprising the drug formulation and/or the hydrogel pushing formulation.

Devices shown and described in International Patent Application No. WO2015/19584, entitled “METHOD AND DEVICES FOR TREATING POSTERIOR OCULAR DISORDERS,” and U.S. Pat. No. 9,180,047, entitled “APPARATUS AND METHODS FOR OCULAR INFECTION,” each of which is incorporated herein by reference in its entirety, can be used to perform the SCS injection portion of the methods provided herein.

Any composition or drug formulation suitable for ocular delivery may be injected using any suitable injector of the types shown and described herein. Any of the methods described herein can be performed use any suitable injector of the types shown and described herein. In this manner, the benefits of the targeted drug delivery via the methods and compositions provided herein can be realized.

In some embodiments, the methods and systems provided herein comprise targeted delivery of a drug to a desired location in the eye, wherein the delivered volume is at least about 20 μL, at least about 50 μL, at least about 100 μL, at least about 200 μL or at least about 500 μL. In some embodiments, the delivered volume is from about 10 μL to about 200 μL, e.g., from about 50 μL to about 150 μL. In some embodiments, the delivered volume is about 50 μL, about 75 μL, about 100 μL, about 125 μL, about 150 μL, about 175 μL, about 200 μL, about 250 μL, about 300 μL, about 350 μL, about 400 μL, or about 500 μL.

In some embodiments, the targeting of a drug formulation to and/or within the posterior segment of the eye is measured by determining how much of the drug formulation (e.g., by volume or by percent) is localized at least 3 mm posterior to the limbus, at least 4 mm posterior to the limbus, at least 5 mm posterior to the limbus, at least 6 mm posterior to the limbus, at least 7 mm posterior to the limbus, at least 8 mm posterior to the limbus, at least 9 mm posterior to the limbus, or at least 10 mm posterior to the limbus. In some embodiments, the targeting of a drug formulation and/or within the posterior segment of the eye is measured by determining how much of the drug formulation (e.g., by volume or by percent) is localized about 0-3 mm posterior to the limbus, about 3-6 mm posterior to the limbus, about 6-9 mm posterior to the limbus, or about 9 or more mm posterior to the limbus.

### Hydrogel Pushing

In some embodiments, the present disclosure provide methods for delivery in the SCS using a hydrogel pushing strategy. In some embodiments, the hydrogel pushing strategy involves a two-step process or a two-formulation process. For example, in some embodiments, two different formulations are introduced (e.g., injected) into the SCS. One of the formulations is the “drug formulation” (used interchangeably herein with “particle formulation,” “drug particle formulation,” “active agent,” “active agent formulation,” and the like) and the other is the “pushing formulation” (referred to herein in some embodiments, as the “hydrogel formulation,” “hydrogel pushing formulation,” or the like). The drug formulation contains whatever materials are to be delivered to specific locations(s) in the SCS in a suitable carrier fluid (e.g., water) and containing suitable excipients (if needed). The pushing formulation may also contain the same or different materials to be delivered to specific locations(s) in the SCS, or may contain no such materials.

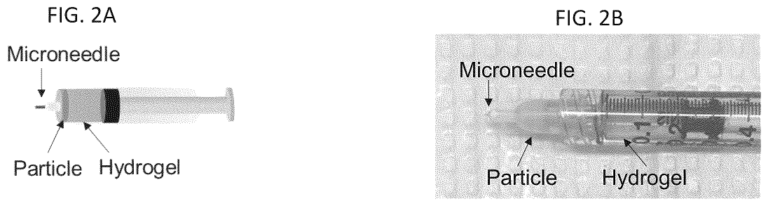

The drug and pushing formulations are introduced into the SCS substantially at the same time or are introduced into the SCS separately. For example, in some embodiments, the drug formulation and the pushing formulation are both loaded into the same microneedle device or microneedle coupled to a syringe. For example, formulations loaded into a microneedle coupled to a syringe are shown in schematically in FIG. 2A and by photograph in FIG. 2B. The “particle” in FIGS. 2A and 2B refers to a drug formulation that comprises a drug particle. In some embodiments, the drug particle formulation is located proximal to the needle portion of the microneedle and between the needle portion of the microneedle and the hydrogel formulation as shown in FIGS. 2A and 2B.

Through a single pressing of the syringe, the drug formulation can be injected into the SCS, with the pushing formulation following immediately behind (i.e., into the SCS). In some embodiments, the drug and pushing formulations are in the opposite order (i.e., drug formulation toward the plunger end). Thus, in some embodiments, the pushing formulation is administered to the SCS first and the drug formulation follows. In such embodiments, the drug formulation may be pushed anteriorly rather than posteriorly. In some embodiments, there are multiple regions within the syringe containing drug or pushing formulations. In some embodiments, the drug and pushing formulations could be introduced at substantially different times using the same or multiple syringes.

Once injected into the SCS, the drug and pushing formulations will occupy substantially different regions of the SCS. In some embodiments, over time, the pushing formulation expands in volume, which pushes at least a portion of the drug formulation to a different region of the SCS. This expansion in volume and further pushing is referred to herein as “spreading,” “hydrogel spreading,” “hydrogel swelling,” “pushing formulation spreading,” “pushing formulation swelling,” and the like. The pushing formulation may push the drug formulation toward specific targeted region(s) of the eye (e.g., to promote desired effects) and/or away from specific targeted region(s) of the eye (e.g., to prevent undesired effects). The timescale of pushing and/or spreading can substantially occur within minutes, hours, days, weeks, or longer, depending on the formulations used. In some embodiments, the pushing formulation continues to swell and push the first formulation toward the posterior segment for at least 1 hour, at least 2 hours, at least 3 hours, at least 4 hours, at least 5 hours, at least 6 hours, at least 7 hours, at least 8 hours, at least 9 hours, at least 10 hours, at least 18 hours, at least 24 hours, or more. In some embodiments, the first formulation, following swelling, remains to the further posterior position for at least 1 day, at least 2, days, at least 3 days, at least 4 days, at least 5 days, at least 6 days, at least 7 days, at least 10 days, at least 2 weeks at least 3 weeks, at least 4 weeks, or longer after injection. In some embodiments, the swelling of the pushing formulation occurs as the formulation expands due to uptake of water from the eye tissue. In some embodiments, the swelling of the pushing formulation is further enhanced when the pushing formulation comprises a salt to drive osmotic flow into the formulation.

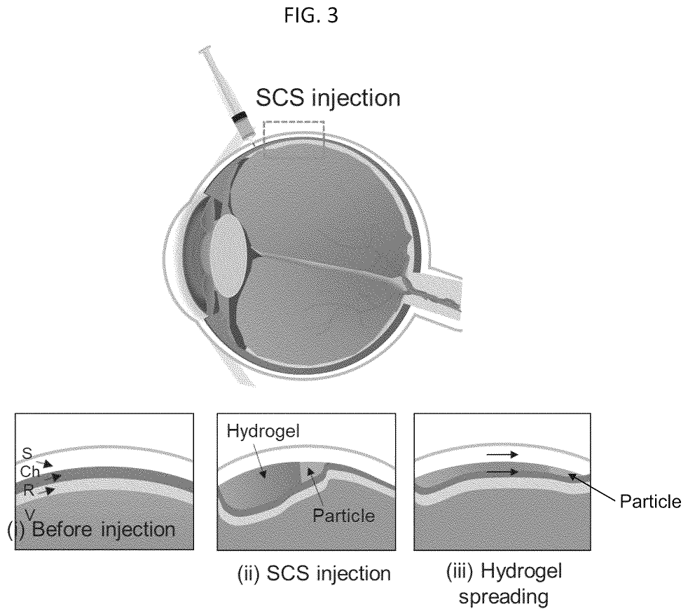

FIG. 3 schematically shows the concept of the hydrogel pushing method before injection, during and after SCS injection, and the effect of the hydrogel spreading after injection. The formulation infuses into the SCS, where the drug particle is first injected. After injection into the SCS, the hydrogel swells, or pushes drug further toward the back of the eye, as shown in FIG. 3.

The drug and pushing formulations are designed according to a number of criteria. For example, in some embodiments, the formulations are biocompatible with the eye and satisfy other safety concerns. In some embodiments, the formulations are compatible with the active agents or drugs they deliver to the eye. In some embodiments, the formulations have a viscosity (e.g., dynamic viscosity) during injection low enough that allows them to flow into the SCS. In some embodiments, the viscosity is low enough to allow flow within the SCS during the pushing. In certain embodiments, the drug and pushing formulations do not substantially mix with each other. In one aspect, the goal of the hydrogel pushing procedure is for a separate pushing formulation to push the separate drug formulation and not have them substantially mix before or during the pushing. However, some mixing is acceptable, as long as the pushing formulation pushes at least a portion of the drug formulation to a different region of the SCS. The separation of the drug and pushing formulations can be due to thermodynamic and/or kinetic reasons. Thermodynamic reasons include the use of formulations that are at least substantially immiscible through use, for example, of different solvents/carrier fluids. Kinetic reasons include high viscosity that slows the mixing of the formulations.

In one aspect, the drug formulation has a lower viscosity than the pushing formulation during injection into the SCS and/or during the pushing within the SCS. This enables the pushing formulation to apply a force to the drug formulation while reducing the mixing of the two formulations. Pushing is achieved by swelling the pushing formulation. Swelling can be achieved by a number of different mechanisms and possibly triggered by a number of different mechanisms. The pushing formulation can form a gel that expands due to, for example and without limitation, physicochemical cues, such as change in pH, change in temperature, change in divalent cation concentration and other triggers of gel swell.

In some embodiments, the formulation can undergo a phase change, for example from liquid to gas, for example caused by a temperature change. In some embodiments, the pushing formulation can expand due to influx of other materials into the formulation, such as water from the surrounding tissues and/or the drug formulation. This could be accomplished by osmotic forces caused by higher solute content in the pushing formulation compared to surrounding fluids. In some embodiments, the formulations in a single syringe can comprise a drug particle phase and a swellable pushing hydrogel phase. The formulations to be injected in a single syringe can be a single or multiple formulations.

In some embodiments, the drug formulation can contain drugs, molecules, particles, hydrogels, polymers, diagnostics, therapeutics and other agents (materials). In some embodiments, the drug particle phase can have a positive or a negative charge, or no charge, and if zwitterionic, a net positive charge, net negative charge or no net charge without modifications.

In some embodiments, the pushing formulation can contain drugs, molecules, particles, hydrogels, polymers, diagnostics, therapeutics and other agents (materials) and it can have any charges or no charge, and if zwitterionic, a net positive charge, net negative charge or no net charge. In some embodiments, the pushing formulation comprises swellable or non-swellable materials. In some embodiments, the pushing formulation comprises or is a gel or an oil, such as silicone oil. In certain embodiments, the pushing formulation has a higher density relative to the drug formulation. In some embodiments, the drug formulation and pushing formulation are a single formulation having the drug or other material for delivery to the desired ocular tissue and having one or more characteristics that allow hydrogel pushing and/or swelling to push the entire formulation toward the posterior segment of the eye after injection.

In some embodiments, the drug formulation comprises particles. The particles of the drug formulations provided herein may be drug, or may be particles that contain drug. In some embodiments, the active agent or drug has a charge. In some embodiments, the active agent or drug has a positive charge or a negative charge. In some embodiments, the active agent or drug is within or associated with particles that have a charge. In some embodiments, the particles have a positive charge or a negative charge. In one embodiment, the drug formulation includes microparticles or nanoparticles, either of which includes at least one drug. Desirably, the microparticles or nanoparticles provide for the controlled release of drug into the suprachoroidal space and surrounding posterior ocular tissue. As used herein, the term “microparticle” encompasses microspheres, microcapsules, microparticles, and beads, having a number average diameter of from about 1 μm to about 100 μm, for example from about 1 to about 25 μm, or from about 1 μm to about 7 μm. “Nanoparticles” are particles having an average diameter of from about 1 nm to about 1000 nm. The microparticles, in one embodiment, have a D₅₀ of about 3 μm or less. In a further embodiment, the D₅₀ is about 2 μm. In another embodiment, the D₅₀ of the particles in the drug formulation is about 2 μm or less. In another embodiment, the D₅₀ of the particles in the drug formulation is about 1000 nm or less. In one embodiment, the drug formulation comprises microparticles having a D₉₉ of about 10 μm or less. The microparticles, in one embodiment, have a D₅₀ of about 3 μm or less. In a further embodiment, the D₅₀ is about 2 μm. In another embodiment, the D₅₀ of the particles in the drug formulation is about 2 μm or less. In another embodiment, the D₅₀ of the particles in the drug formulation is about 1000 nm or less. In one embodiment, the drug formulation comprises microparticles having a D₉₉ of about 10 μm or less. The microparticles, in one embodiment, have a D₅₀ of about 3 μm or less. In a further embodiment, the D₅₀ is about 2 μm. In another embodiment, the D₅₀ of the particles in the drug formulation is about 2 μm or less. In another embodiment, the D₅₀ of the particles in the drug formulation is about 100 nm to about 1000 nm. In one embodiment, the drug formulation comprises microparticles having a D₉₉ of about 1000 nm to about 10 μm. The microparticles, in one embodiment, have a D₅₀ of about 1 μm to about 5 μm or less. In another embodiment, the drug formulation comprises particles having a D₉₉ of about 10 μm. In another embodiment, the D₉₉ of the particles in the formulation is less than about 10 μm, or less than about 9 μm, or less than about 7 μm or less than about 3 μm. The microparticles or nanoparticles, in some embodiments, have a charge, such as a negative charge.

Microparticles and nanoparticles may or may not be spherical in shape. “Microcapsules” and “nanocapsules” are defined as microparticles and nanoparticles having an outer shell surrounding a core of another material. The core can be liquid, gel, solid, gas, or a combination thereof. In one case, the microcapsule or nanocapsule may be a “microbubble” or “nanobubble” having an outer shell surrounding a core of gas, wherein the drug is disposed on the surface of the outer shell, in the outer shell itself, or in the core. (Microbubbles and nanobubles may be respond to acoustic vibrations as known in the art for diagnosis or to burst the microbubble to release its payload at/into a select ocular tissue site.) “Microspheres” and “nanospheres” can be solid spheres, can be porous and include a sponge-like or honeycomb structure formed by pores or voids in a matrix material or shell, or can include multiple discrete voids in a matrix material or shell. The microparticles or nanoparticles may further include a matrix material. The shell or matrix material may be a polymer, amino acid, saccharride, or other material known in the art of microencapsulation.

The drug-containing microparticles or nanoparticles may be suspended in an aqueous or non-aqueous liquid vehicle. The liquid vehicle may be a pharmaceutically acceptable aqueous solution, and optionally may further include a surfactant. The microparticles or nanoparticles of drug themselves may include an excipient material, such as a polymer, a polysaccharide, a surfactant, etc., which are known in the art to control the kinetics of drug release from particles.

In some embodiments, the pushing formulation comprises hyaluronic acid (HA). In some embodiments, both the drug formulation and the pushing formulation comprise HA. In some embodiments, the pushing formulation comprises a higher % (w/v) HA than the drug formulation. In some embodiments, the drug formulation comprises 0% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2.0% (w/v), about 2.5% (w/v), about 3.0% (w/v), about 3.5% (w/v), about 4.0% (w/v), about 4.5% (w/v), about 5.0% (w/v) HA or more. In some embodiments, the pushing formulation comprises about 1% (w/v), about 1.5% (w/v), about 2% (w/v), about 2.5% (w/v), about 3% (w/v), about 3.1% (w/v), about 3.2% (w/v), about 3.3% (w/v), about 3.4% (w/v), about 3.5% (w/v), about 3.6% (w/v), about 3.7% (w/v), about 3.8% (w/v), about 3.9% (w/v), about 4.0% (w/v), about 4.1% (w/v), about 4.2% (w/v), about 4.3% (w/v), about 4.4% (w/v), about 4.5% (w/v), about 4.6% (w/v), about 4.7% (w/v), about 4.8% (w/v), about 4.9% (w/v), about 5.0% (w/v), about 5.5% (w/v), about 6.0% (w/v), about 6.5% (w/v), about 7.0% (w/v), about 7.5% (w/v), about 8.0% (w/v), about 8.5% (w/v), about 9.0% (w/v), about 10% (w/v), or more HA. In some embodiments, the drug formulation comprises about 0.1% (w/v) to about 2.5% (w/v) HA and the pushing formulation comprises about 2.5% (w/v) to about 5% (w/v) HA. In some embodiments, the drug formulation comprises about 0.5% (w/v) to about 1.5% (w/v) HA and the pushing formulation comprises about 3.0% (w/v) to about 5% (w/v) HA. In some embodiments, the drug formulation comprises about 1% (w/v) HA and the pushing formulation comprises about 4% (w/v) HA.

In some embodiments, one or more of the formulations comprises a salt. For example, in some embodiments, one or more of the formulations (e.g., the drug formulation or the pushing formulation) comprises a salt selected from the group consisting of NaCl, MgCl, MnCl₂, CaCl₂, ZnCl₂, and KCl. In some embodiments, the pushing formulation is a high-salt formulation. In some embodiments, the pushing formulation comprises about 5%, about 6%, about 7%, about 8%, about 9%, about 10%, about 11 %, about 12%, about 13%, about 14%, or about 15% NaCl, or any other salt known in the art. For example, in some embodiments, the pushing formulation comprises about 9% NaCl. In some embodiments, increasing the salt concentration of the pushing formulation results in a pushing formulation that exhibits enhanced, increased, and/or more rapid pushing of the drug formulation further posteriorly in the posterior segment of the eye.

In some embodiments, the total volume injected into the suprachoroidal space of the eye (inclusive of drug formulation and pushing formulation) is about 10 μL, about 20 μL, about 30 μL, about 40 μL, about 50 μL, about 60 μL, about 70 μL, about 80 μL, about 90 μL, about 100 μL, about 110 μL, about 125 μL, about 150 μL, about 175 μL, about 200 μL, about 225 μL, about 250 μL, about 300 μL, about 400 μL, or about 500 μL. In some embodiments, the drug formulation is present at an equal, a higher, or a lower amount relative to the pushing formulation. For example, in some embodiments, the total injection volume is 50 μL, the volume of the drug formulation 20 μL, and the volume of the pushing formulation 30 μL.

In some embodiments, the order of the formulations in a single syringe can be altered depending on the objective. For example, the drug particle formulation may be closer to the microneedle end and the pushing formulation may be distal to the microneedle end. In some embodiments, the drug particle formulation can be delivered to a greater extent toward anterior or posterior area depending on the sort and order of the formulations in a single syringe. This procedure can be conducted on any eye that contains, or can be made to contain, a suprachoroidal space, including human and animal eyes for medical, veterinary and other applications.

### Iontophoresis

In one aspect, the present disclosure provides methods for targeted delivery to tissues within the eye of a subject in need thereof, comprising SCS injection and iontophoresis application. In some embodiments, for delivery in the SCS using iontophoresis, a two-step process is provided. In the first step, a drug formulation is introduced (e.g., injected) into the SCS. The drug formulation contains whatever materials are to be delivered to specific locations(s) in the SCS in a suitable carrier fluid (e.g., water) and containing suitable excipients (if needed). In the second step, an electric field is applied such that charged species move within the SCS by iontophoresis. “Tontophoresis” herein includes electrophoresis, electoosmosis, electrically enhanced diffusion and other electrokinetic phenomena.

In some embodiments, the drug formulation is be injected and iontophoresis applied into the SCS substantially at the same time. In other embodiments, the drug formulation is injected and iontophoresis is applied separately or concurrently. For example, if introduced at substantially the same time, the drug formulation in some embodiments is injected into the SCS and iontophoresis is applied before, during and/or after the injection. If introduced at substantially different times, the drug formulation could be injected in the absence of iontophoresis, and iontophoresis could be applied after the injection is finished.

In one aspect, using this approach, the region(s) of the SCS occupied by the drug formulation targeted using iontophoresis is different from that which would have been occupied if the same procedure had been followed but without using iontophoresis. If the iontophoresis is applied after the drug formulation is injected, then the drug formulation will occupy a different region of the SCS after the application of iontophoresis compared to before iontophoresis. A “different region” means that at least a portion of the drug formulation occupies a region of the eye that is different from the comparator region (e.g., without or before iontophoresis). The timescale of iontophoresis application can occur for seconds, minutes or possibly hours, depending on the conditions used and the objectives of the procedure.

In some embodiments, the drug formulation for use in the iontophoresis method contains materials to be delivered to specific locations(s) in the SCS. Materials may be charged (positively and/or negatively) with a net charge or a net neutral charge (e.g., zwitterionic) and/or they may be uncharged. Materials may be contained within carrier materials such as particles like nanoparticles, microparticles, liposomes, micelles, colloids and other structures. The carrier materials (used interchangeably herein with “containing materials”) may be charged (positively and/or negatively) with a net charge or a net neutral charge (e.g., zwitterionic) and/or they may be uncharged. The charge on the materials and the charge on the carrier materials influence the movement of the drug formulation in the SCS during iontophoresis. For example, if the net charge on the materials or on the carrier materials is negative, then the materials and the carrier materials will be moved away from the negative electrode and toward the positive electrode. As another example, if the net charge on the material or on the carrier materials is positive, then the materials and the carrier materials will be moved away from the positive electrode and toward the negative electrode. If there is no charge or no net charge on the material or on the carrier materials, then the materials and the carrier materials can be moved by electroosmosis. Electrically enhanced diffusion can be achieved, for example, by applying an alternating the electric field and/or alternating the current.

In one aspect, iontophoresis is applied by using at least two electrodes that are capable of generating an electric field within the SCS. For example, an electrode can be associated with the syringe used to inject the drug formulation. An electrode could be the needle, or part of the needle or attached to the needle used to inject the drug formulation. An electrode could be located within the barrel of the syringe, attached to the inner wall of the syringe, the plunger or another part of the syringe. In some embodiments, the electrode is located within the syringe, for example on the end of the plunger, and the wire is passed through the rubber stopper of the syringe. In some embodiments, an electrode is physically separate from the syringe and/or needle used for injection of the drug formulation. In some embodiments, there is an electrically conductive pathway from an electrode to the needle and/or syringe and/or through the needle and/or syringe. In some embodiments, there is a conductive pathway from an electrode to at least a portion of the SCS. In some embodiments, there is a conductive pathway from an electrode to and through the site of injection of materials into the SCS. Typically an electrode associated with the injection site would have a polarity (i.e., negative or positive electrode) that repels the materials and/or carrier materials, but not necessarily, depending on the location to which the materials are being targeted.

In some embodiments, an electrode is located distant from the injection site. In some embodiments, an electrode located distant from the injection site has a polarity (i.e., negative or positive electrode) that attracts the materials and/or carrier materials. In some embodiments, the distant location would typically be somewhere on the head or neck, possibly the ear or forehead or other skin surface on the head or neck. The distant location could be within the head, such as within the mouth or nose. The skin could be prepared with an conductive gel, by sandpapering the skin or other methods known in the art to facilitate good electrical contact between an electrode and skin and to reduce the electrical resistance of the skin.

In some embodiments, a conductive material is applied at a site of an electrode to facilitate passage of current and/or heat transfer. For example, in some embodiments, an electrically conductive material is used to increase the effective contact area of an electrode with the skin, eye or other tissue it contacts to reduce the current density for a given current. For example, a thermally conductive material is used to facilitate heat transfer away from an electrode and/or contact area with the skin, eye or other tissue it contacts to reduce heating at those sites.

Without an iontophoresis application, the materials injected using a microneedle localize in the suprachoroidal space, with precise location depending on the material, the formulation, the injection procedure and other parameters. The distribution of the injected materials can be changed with application of an iontophoresis. For example, in some embodiments, the injected material can be delivered to a greater extent toward one or the other of the electrodes, depending on the charge of the material, the electrode polarity and other factors (e.g., positively charged materials will move away from the positive electrode and toward the negative electrode). Movement caused by the current can be by electrophoresis, electroosmosis, electromigration, iontophoresis or other phenomena that move charged and/or uncharged materials due to an electric field or current. In this manner, precise targeting to specific tissues and areas can be achieved.

In one aspect, the electric field or current is applied by controlling the voltage, controlling the current, or both. In some embodiments, a constant voltage is maintained. In some embodiments, a constant current is maintained. In other embodiments, the voltage and/or current is changed over time.

The electric field and/or current is selected by balancing multiple parameters including sufficient force to move materials to a desired location(s), and preferably not to cause damage, pain, side effects or other adverse effects to the eye, the body, or the operator. In some embodiments, the iontophoresis is applied using a current having an absolute value of about 0.01 mA, about 0.05 mA, about 0.10 mA, about 0.15 mA, about 0.2 mA, about 0.3 mA, about 0.4 mA, about 0.5 mA, about 0.6 mA, about 0.7 mA, about 0.8 mA, about 0.9 mA, or about 1.0 mA. The polarity of the current is selected depending on the net charge of the drug or particles and the location to which the drug or particles is to be targeted (e.g., anteriorly or posteriorly to the injection site) as described herein. In some embodiments, the iontophoresis is applied using a positive current of about 0.01 mA, about 0.05 mA, about 0.10 mA, about 0.15 mA, about 0.2 mA, about 0.3 mA, about 0.4 mA, about 0.5 mA, about 0.6 mA, about 0.7 mA, about 0.8 mA, about 0.9 mA, or about 1.0 mA, and the method is performed using particles or active agent having a net negative charge. In other embodiments, the iontophoresis is applied using a negative current of about 0.01 mA, about 0.05 mA, about 0.10 mA, about 0.15 mA, about 0.2 mA, about 0,3 mA, about 0.4 mA, about 0.5 mA, about 0.6 mA, about 0.7 mA, about 0.8 mA, about 0.9 mA, or about 1.0 mA, and the method is performed using particles or active agent having a net negative charge. In still other embodiments, the polarity of the current and the drug or particles is the same.

In one aspect, the iontophoresis methods provided herein can be conducted on any eye that contains, or can be made to contain, a suprachoroidal space, including human and animal eyes for medical, veterinary and other applications.

In some embodiments, the electrodes are designed to minimize pH changes during application of the electric field and/or current, because pH changes can damage tissue, can alter the charge on materials and tissue and can have other effects that influence safety and efficacy of the procedure. In some embodiments, an electrode can be associated with a needle and/or syringe. In one embodiment, the needle itself can be an electrode (e.g., an electrically conductive needle). In another embodiment, the electrode can be placed in the interior of the syringe, for example in fluid and/or electrical communication with the contents of the syringe. The electrode can be attached to the plunger in the syringe or to the side-wall of the syringe. In one embodiment, the electrode can be placed around the microneedle so that the electrode can attach on the eye tissues or not eye tissues in the electric communication via the microneedle or eye tissues or the injection hole.

An electrode can be in electrical communication with a tissue of the eye and/or a tissue not part of the eye. In some embodiments, the electrode is in electrical communication with a surface of the eye, such as the cornea, or conjunctiva, or sclera, or in the periocular space. In some embodiments, the electrode is in electrical communication with a surface not of the eye, such as the skin or the oral mucosa. For example, the skin could be on the head, the face, the ear (e.g., the ear lobe), the nose, the forehead, or the temple. In some embodiments, the site of electrical communication with an electrode could be treated to facilitate good electrical contact with the body, such as by application of electrically conductive gel and abrasion of the site (e.g., abrasion with sandpaper). In one aspect, the present disclosure provides a kit for targeted delivery of a substance to a region of the eye, wherein the kit comprises the electrodes, microneedles, and syringes provided herein.

### Combined Hydrogel Pushing and Iontophoresis

In one aspect, the present disclosure provides compositions, systems, methods, devices, and kits for drug administration to targeted portions of the eye using a combination of hydrogel pushing and iontophoresis. Precise delivery to target tissues and areas can be modulated by using SCS injection and a combination of both the hydrogel pushing and iontophoresis approaches described herein. The various events-injection of drug formulation, injection of pushing formulation, application of iontophoresis-can be done all at the same time, two at the same time or none at the same time. For example, in some embodiments, the drug formulation and pushing formulation are performed concurrently or sequentially, and iontophoresis is applied following the injection of the drug and pushing formulations. In other embodiments, the iontophoresis may be applied before or during injection of the drug and/or pushing formulations. In such embodiments, the drug formulation maybe be pushed by the pushing formulation and the iontophoresis application further posteriorly in the eye. In still other embodiments, the pushing formulation may be administered first, followed by concurrent or sequential administration of the drug formulation and iontophoresis application. In certain embodiments, a drug formulation is administered by SCS injection using a microneedle, iontophoresis is applied after injection of the drug formulation, and a pushing formulation is injected into the SCS using a microneedle after application of iontophoresis.

The present invention is further illustrated by reference to the following Examples. However, it should be noted that these Examples, like the embodiments described above, are illustrative and are not to be construed as restricting the scope of the invention in any way.

### EXAMPLES

### Example 1. Targeted Suprachoroidal Space Drug Delivery Using Hyaluronic Acid Hydrogel Pushing

A study was conducted to assess the targeting of drug delivery by hydrogel pushing in conjunction with injection into the suprachoroidal space of the eye and to study the effects of varied formulation characteristics in the methods provided herein. Drug particle formulations comprising red fluorescent particles and 0%, 1%, 2%, or 4% HA were prepared. The drug particle formulations at a volume of 20 μL were loaded into a microneedle with a 4% HA pushing formulation at a volume of 30 μL, and the formulations were infused into the SCS of rabbit eyes ex vivo. Immediately after injection of the formulations, the ex vivo eye was frozen and dissected to assess the extent to which the various formulations pushed to the posterior pole of the eye.

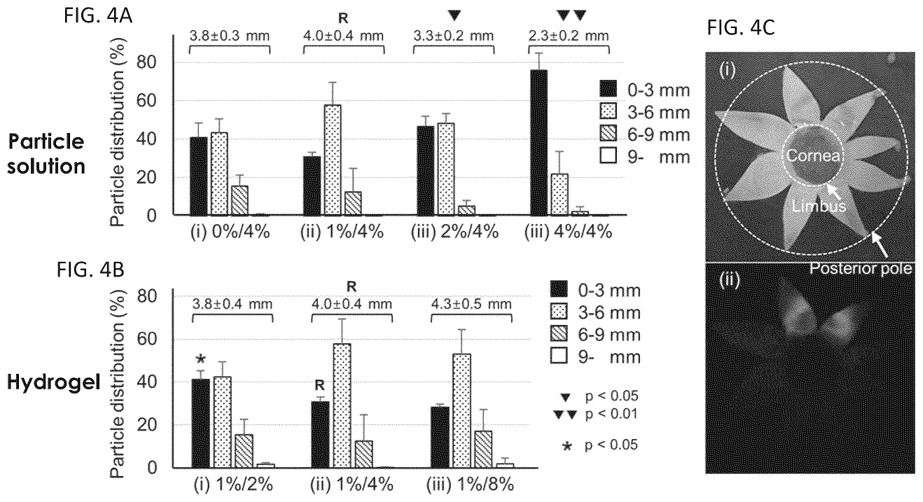

FIG. 4A shows the distributions of the red-fluorescent particles after SCS injection of each of the 4 formulations. The figure shows the percent of particles distributed to the increasingly distant regions as indicated (0-3 mm, 3-6 mm, 6-9 mm, or 9 mm or more). The averaged particle transfer (APT) for each group is also shown at the top of the bars for each of the groups in FIG. 4A. The 0% HA drug particle formulation had an APT of 3.8±0.3 mm; the 1% HA drug particle formulation had an APT of 4.0±0.4 mm; the 2% HA drug particle formulation had an APT of 3.3±0.2 mm; and the 4% HA drug particle formulation had an APT of 2.3±0.2 mm. Accordingly, decreasing concentrations of HA resulted in less pushing of the particle formulation to the posterior pole, but some amount of HA present in the drug particle formulation was required for optimal results. The study showed that the particle formulation requires a small amount of viscosity to be optimally pushed by the pushing formulation. In summary, the formulation with 1% was chosen as the drug particle solution for further studies.

FIG. 4B shows the particle distribution (% of particles located at the indicated positions) when the drug particle formulation was held at 1% HA and the pushing formulation was tested at 2%, 4%, or 8% HA. The pushing effect was improved with 4% or 8% HA relative to 2% HA. The APT for 2% HA in the pushing formulation was 3.8±0.4 mm; the APT for 4% HA in the pushing formulation was 4.0±0.4 mm; and the APT for 8% HA in the pushing formulation was 4.3±0.5 mm. The 8% HA formulation exhibited many bubbles due to the high viscosity. Accordingly 4% HA was selected as the concentration for the pushing formulation for further studies.

FIG. 4C shows a digital (i) and fluorescent (ii) image following injection of the formulation comprising 1% HA in the drug particle formulation and 4% HA in the pushing formulation (“1%/4% formulation”). The red fluorescent particles in the drug formulation was pushed backward from the injection site to the posterior pole.

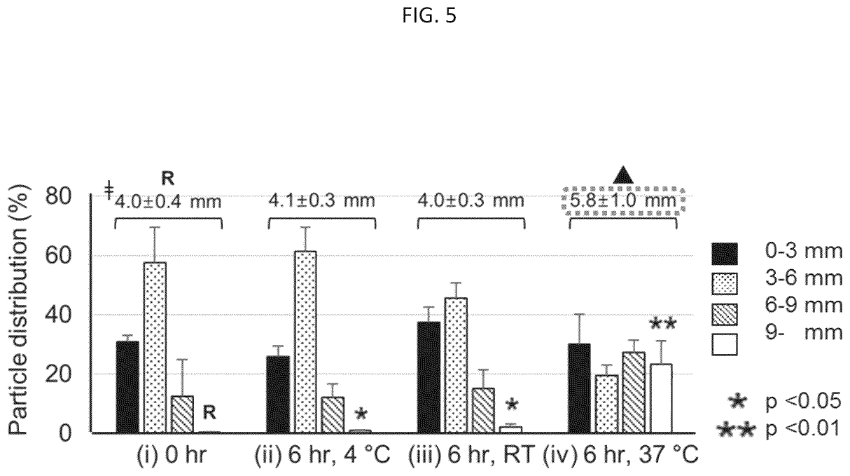

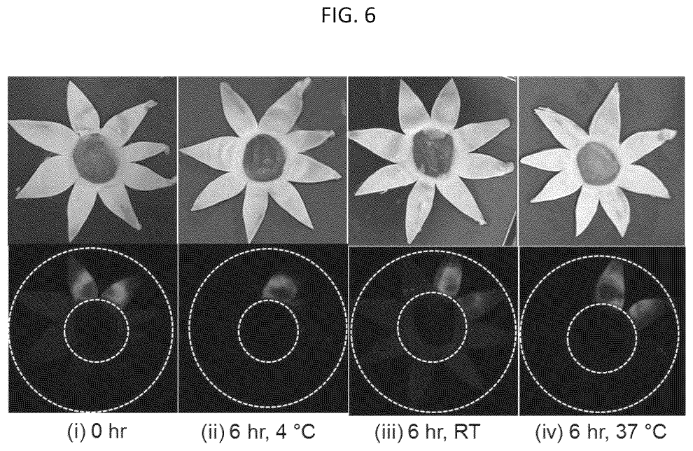

Next, the effects of incubation at different temperatures on the particle delivery toward the back of the eye was assessed. The 1%/4% formulation was injected in a 50 μL volume into the SCS of rabbit eyes ex vivo and eyes were incubated for 0 hr, 6 hrs at 4° C., 6 hrs at room temperature (RT), or 6 hrs at 37° C. Distributions of the red fluorescent particles in the SCS in each condition are shown in FIG. 5. Particle distribution was similar in the 0 hr (i) and 4° C. (ii) groups. At RT (iii), a small amount of the particles were moved to the back of the eye. When the incubation temperature was 37° C. (iv), the drug particle was localized around the optic nerve. The APT for the 0 hour incubation (i) was 4.0±0.4 mm; the APT for the incubation at 4° C. was 4.1±0.3 mm; the APT for the incubation at RT was 4.0±0.3 mm; and the APT for the incubation at 37° C. was 5.8±1.0 mm. FIG. 6 shows the bright field (top row) and fluorescent (bottom row) images of the rabbit eyes ex vivo with each of the incubation conditions. The particles were transferred to the back of the eye in the 6 hour, 37° C. incubation group.

The study showed that using the 1%/4% formulation, 14.1±5.4% of the particles was delivered toward the back of the eye (i.e., >6 mm posterior to the limbus) right after injection; and 54.3±10.2% of particles were delivered >6 mm posterior to the limbus after 6 h at 37° C. This demonstrated the ability of the pushing formulation to move more than half of the particles to the back part of the SCS. This pushing was facilitated not only by the increase in hydrogel volume upon contact with water in the eye, but also because the viscosity of the HA pushing hydrogel was decreased at 37° C. (relative to room temperature at which it was injected).

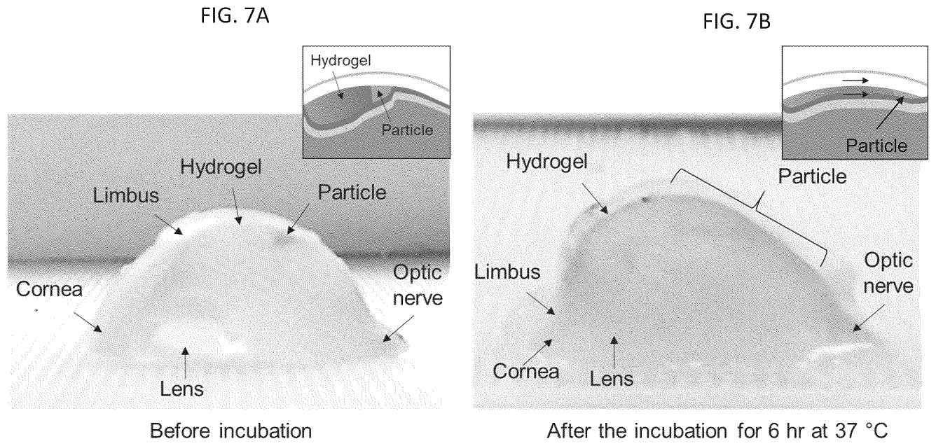

FIG. 7A shows a digital image of a dissected eye right after the injection of the 1%/4% formulation. The drug particle was localized at the end of the SCS by the pushing hydrogel. FIG. 7B shows a digital image of a dissected eye after a 6 hour incubation at 37° C. following the injection. The image shows that after the incubation, the particle was delivered around the optic nerve by the spreading of the hydrogel.

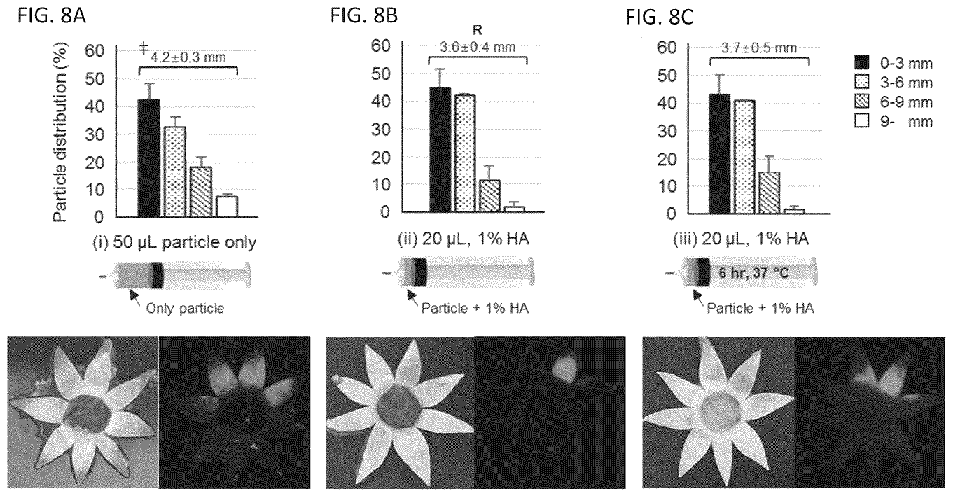

To further assess the effects of the pushing formulation, a study was conducted to observe the effect of injecting various drug particle formulations without a pushing formulation included. FIG. 8A shows the particle distribution (bar graph) and bright field and fluorescent images immediately following SCS injection of 50 μL of red fluorescent particles in HBSS buffer (no HA). This injection achieved only 7.1±1.1% of particles reaching around the back of the eye (9-mm from limbus). FIG. 8B shows the particle distribution (bar graph) and bright field and fluorescent images immediately following SCS injection of 20 μL of particle in 1% HA. FIG. 8C shows the particle distribution (bar graph) and bright field and fluorescent images following SCS injection of 20 μL of particle in 1% HA, after a 6 hour incubation at 37° C. Distributions of the 1% HA and the 1% HA with 6 hour incubation at 37° C. were similar, and there was no significant difference among the groups. Thus, the study showed that without the presence of the pushing formulation, there is no spreading of the drug particle formulation.

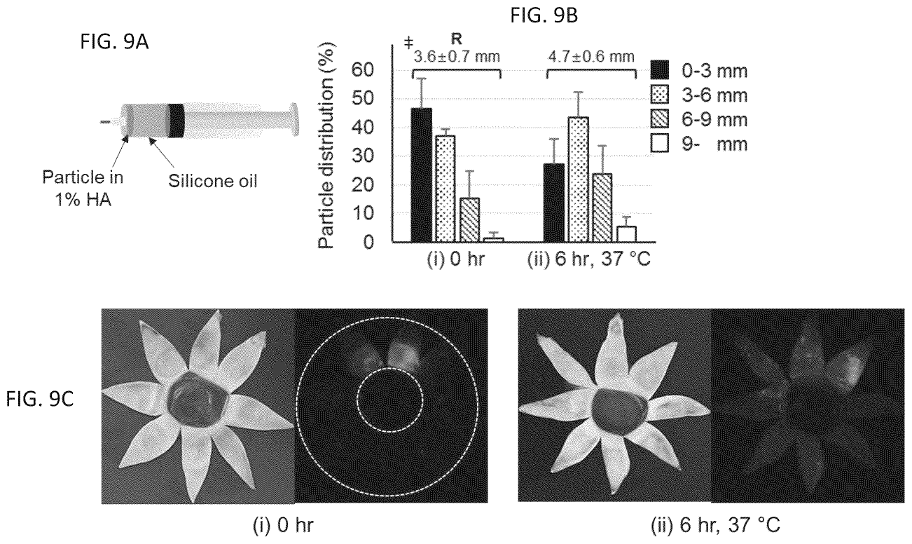

A further study was conducted to assess the ability of a silicone oil formulation to perform the pushing function. Silicone oil was used in place of the HA pushing hydrogel. FIG. 9A is a schematic showing the 1% HA drug particle formulation and silicone oil in the microneedle. FIG. 9B shows the percent particle distribution at the indicated positions immediately after the injection (0 hr) or after a 6 hour incubation at 37° C. following the injection. FIG. 9C shows the bright field and fluorescent images for the two groups. The study showed that the silicone oil did not exhibit the effective particle pushing achieved with the HA pushing formulation. Since the silicone oil is immiscible to the drug particle formulation, the drug was not mixed with the silicone oil in the SCS. However, silicone oil did not exhibit the swelling and pushing effect of the particle to back of the eye. Without wishing to be bound by theory, the lower density of the silicone oil relative to the particle solution may inhibit pushing of the particle solution.

Next, analysis of particle delivery toward the back of the eye using a high salt (9% sodium hydrochloride) HA as the pushing formulation was assessed, to determine whether a high salt HA hydrogel can increase osmotic flow of water into the hydrogel, to increase the pushing effect. 9% sodium chloride was added to the 4% HA pushing formulation, loaded into a microneedle with the red fluorescent drug particle formulation. The formulations were injected into the rabbit eyes ex vivo or in vivo.

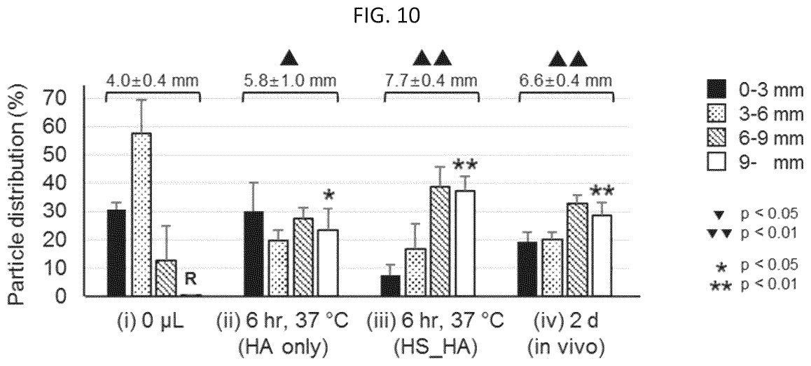

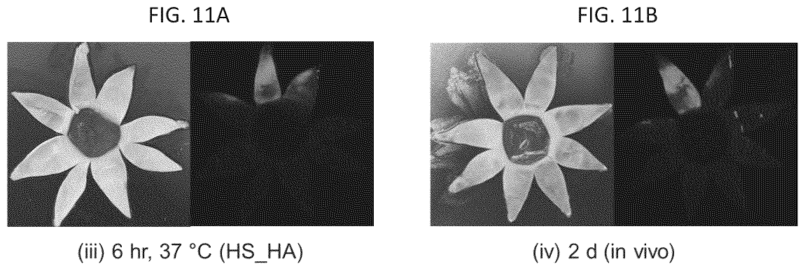

The results of the study are provided in FIG. 10 an FIG. 11A-11C. FIG. 10 shows the particle distribution of red fluorescent particles immediately after ex vivo injection (i), after incubation at 37° C. for 6 hours following the ex vivo injection of the HA hydrogel (without added salt) (ii), after incubation at 37° C. for 6 hours following ex vivo injection of the HA hydrogel with 9% sodium chloride added (iii; “HS_HA”), two days after in vivo injection into rabbit eyes using the high-salt (9% sodium chloride) HA formulation. The normal HA hydrogel delivered 54.3±10.2% of particles over 6 mm posterior to the limbus, and the APT for the normal HA hydrogel was 5.8±1.0 mm. The high-salt HA hydrogel pushed 80.7±6.1% of the particles >6 mm posterior to the limbus (and 38.0±2.1% of particles were >9 mm posterior to the limbus) 6 hours after injection at 37° C., and the APT for this group was 7.7±0.4 mm. Moreover, the in vivo SCS injection of the rabbit eye showed that 61.2±7.7% of the particle was delivered to over 6 mm posterior to the limbus 2 day after the injection, and the APT for this group was 6.6±4.4 mm. FIG. 11A shows the bright field and fluorescent images for the HS_HA group, and FIG. 11B shows the bright field and fluorescent images for the in vivo injection group.

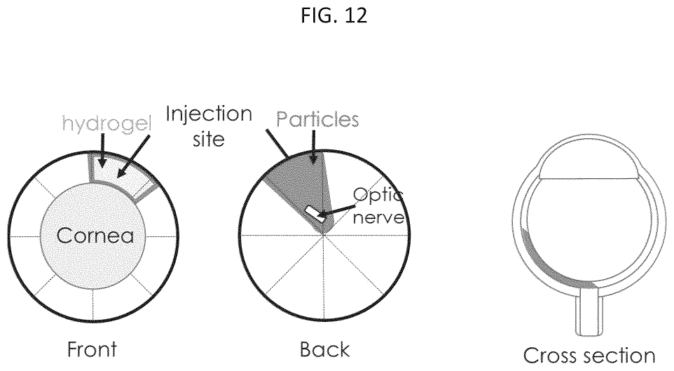

In summary, the study showed that the salts in the HA hydrogel drastically enhanced the pushing effect compared to the normal HA 4% hydrogel pushing formulation without added salt. The drug particles were pushed by the high-salt HA hydrogel formulation such that they were precisely targeted to the back of the eye, as shown schematically in FIG. 12.

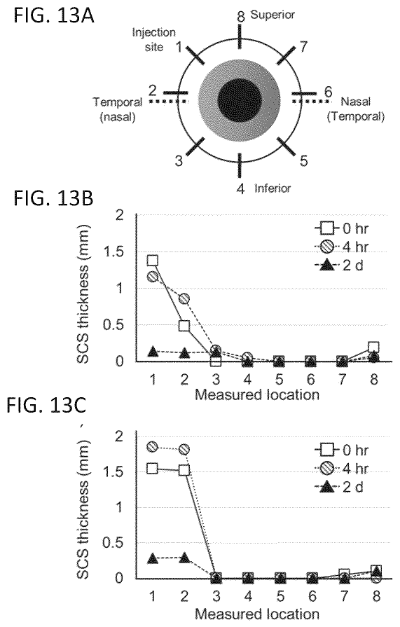

A study was conducted to assess the change in thickness of the SCS upon administration of hydrogel pushing formulations. HA hydrogel and high-salt HA hydrogel formulations were tested. FIG. 13A is a schematic showing the numerical identification of the locations within the eye. The SCS thickness was measured at 8 different areas as shown in FIG. 13A: The injection site is site (1), and the 7 other sites are as shown in the schematic figure. Thickness measurements were conducted using an ultrasound probe, and measurements were performed at three timepoints: 0 hr, 4 hr, and 2 days after the injection. FIG. 13B shows the thickness at each measured location of the SCS, and at each timepoint, after HA hydrogel injection. FIG. 13C shows the thickness at each measured location of the SCS, and at each timepoint, upon high-salt HA hydrogel injection.

The results of the study showed that both formulations resulted in increased thickness; high salt in the HA hydrogel resulted in increased thickness relative to the non-high salt HA hydrogel, particularly at 4 hours after injection. The high-salt HA hydrogel pushing formulation pushed the particles of the first formulation further from the injection site.

### Example 2. Drug Delivery Targeted by Iontophoresis in the SCS of the Eye

Although SCS delivery improves targeting to sites of drug action, further control of drug distribution within the SCS would enable more precise targeting of drug to, for example, the posterior pole. In this Example, studies were conducted to assess whether application of an electric field within the SCS can drive charged molecules and particles, and thereby localize drug delivery to specific sites within the SCS of the eye.

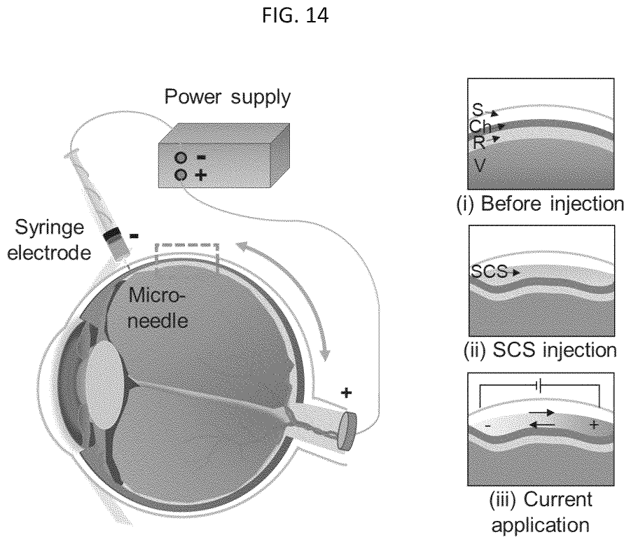

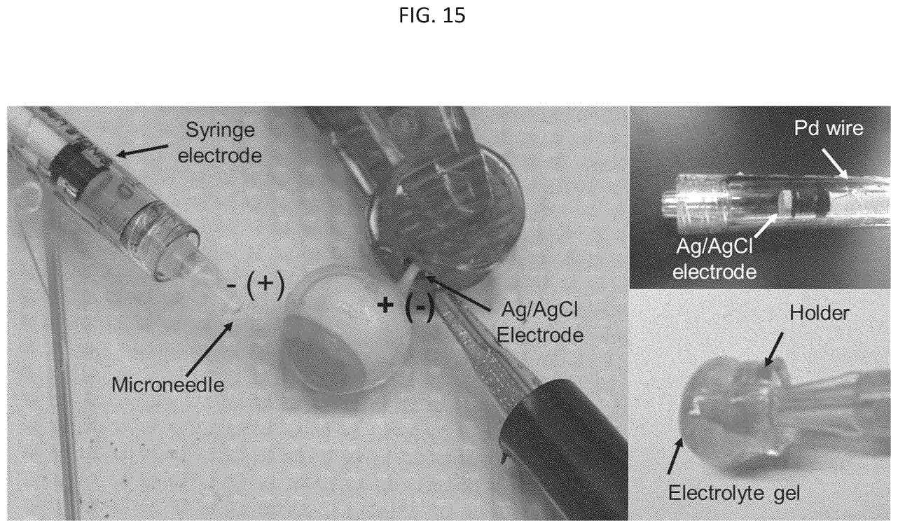

FIG. 14 provides a schematic representation of an exemplary method of iontophoretic targeting for ocular drug delivery via the SCS. A power supply is used to apply an electric field such that charged species delivered into the SCS concurrently or sequentially (e.g., by microneedle injection of a drug formulation into the SCS before, during, or after application of the electric field) move within the SCS by iontophoresis. As shown in box (i) of FIG. 14, before injection, the injection target for the drug formulation is between the sclera (S) and the choroid (Ch) (i.e., the SCS). Upon SCS injection, the SCS is filled with the drug formulation (box (ii) of FIG. 14). A current is applied to cause the charged species to move within the SCS (box (iii) of FIG. 14.). FIG. 15 provides photo images of exemplary materials for use in the iontophoretic microneedle system for injection, for example for injection into a rabbit eye ex vivo. For testing in rabbit eyes ex vivo, the injection syringe contained an electrode (cathode) mounted on the end of the plunger. The other electrode was attached at the end of the optic nerve (anode). A microneedle holder with electrolyte gel was added to facilitate electrical connectivity between the microneedle device and the ocular tissue, and also to reduce the heat generated from the microneedle during iontophoresis application.

New Zealand White rabbit eyes were injected ex vivo with 50 μL, 100 μL, or 200 μL of a formulation comprising red fluorescent particles. The particles were of 20 nm diameter and with a −50 mV zeta potential. The microneedle used was a hollow microneedle with a 30-gauge and 750 μm length. The syringe connected to the microneedle was modified by embedding a Ag/AgCl electrode in the chamber of the syringe, which was connected to a DC power supply. Either no current (i) or 0.14 mA for 3 minutes (ii) was applied. After injection, the eye was snap frozen and cut into 8 scleral petals. Fluorescent images of each eye were acquired, and particle extraction was performed for the distribution analysis. The percent particle distribution at 0-3 mm; 3-6 mm; 6-9 mm; or 9+mm was assessed for each group.

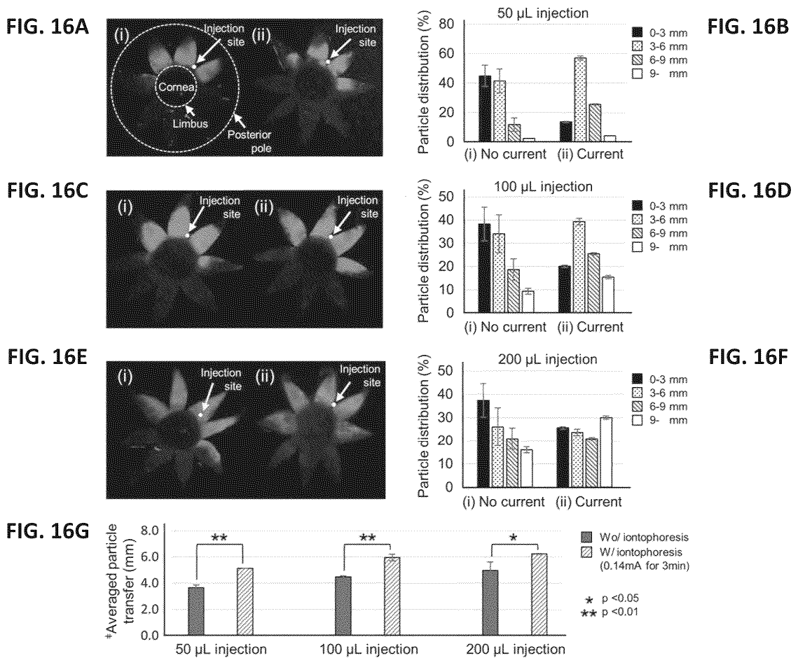

FIGS. 16A, 16C, and 16E show fluorescence micrographs of the distributions of the particles in the SCS after injection with 50 μL, 100 μL, or 100 μL volumes, respectively, and in either the absence (i) or the presence (ii) of iontophoresis. The particle distribution is provided graphically for the same groups in FIGS. 16B, 16D, and 16F for the same groups (50 μL, 100 μL, or 100 μL volumes, respectively, with or without iontophoresis. The average particle transfer (APT) for each group is provided in FIG. 16G. The profile of the APT clearly showed that the iontophoresis increased the particle localization to the posterior region of the eye. The portion of particles in the back of the eye was statistically significantly increased by the application of iontophoresis, as well as by increase in injection volume. With a 200 μL volume injection, 15.9% of particles were found in the most posterior quadrant (9mm+) in the absence of current. However, when iontophoresis was applied (i.e., with current), 30% of the particles were found in the most posterior quadrant.

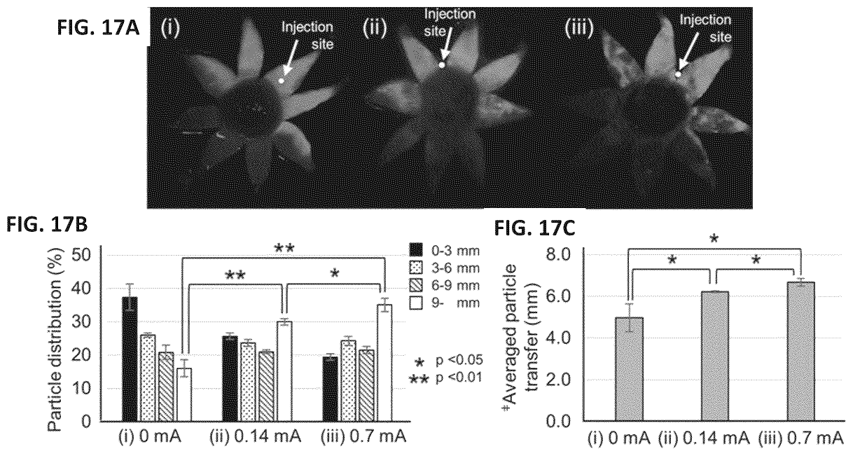

Next, the effect of altering the current on drug particle targeting was assessed. The time of iontophoresis was held at 3 min, and an injection volume of 200 μL was used. Iontophoresis currents of 0 mA, 0.14 mA, and 0.7 mA were tested. FIG. 17A provides the fluorescence micrographs showing the distribution of red-fluorescent particles in the SCS without iontophoresis (0 mA (i)), or with iontophoresis with a current of 0.14 mA (ii) or 0.7 mA (iii). FIG. 17B shows the particle distribution % for each group and FIG. 17C shows the APT for each group. The study showed that particle distribution to the 9+mm area was increased by increasing the current. In addition, the APT data showed that increased current increased the delivered distance of particles. However, added particle delivery was not as enhanced as the current was increased (i.e., 5 fold increase from 0.14 to 0.7 mA). In addition, there was some tissue burn after application of 0.7 mA iontophoresis. 0.14 mA iontophoresis was selected for further studies.

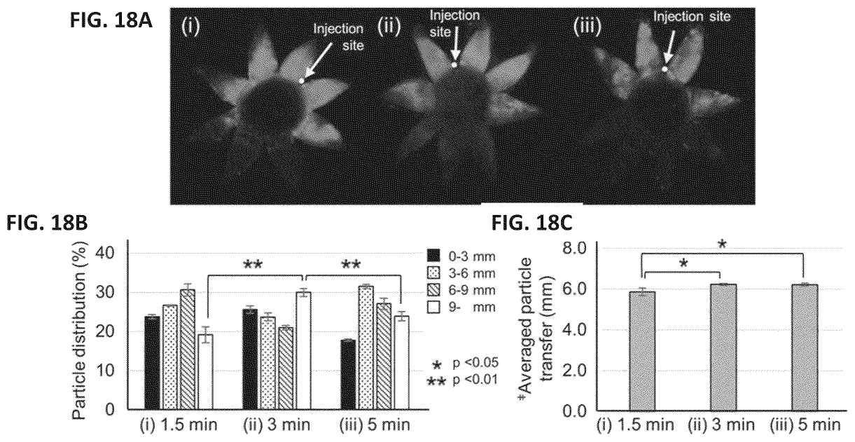

A study was conducted to determine the effect of the application time of iontophoresis. The iontophoresis current was held at 0.14 mA and the injection volume of 200 μL was used. Iontophoresis application times of 1.5 min, 3 min, and 5 min. were compared. FIG. 18A provides the fluorescence micrographs showing the distribution of red-fluorescent particles in the SCS without iontophoresis application times of 1.5 min (i), 3 min (ii), or 5 min (iii). FIG. 18B shows the particle distribution % for each group and FIG. 18C shows the APT for each group. The study showed that particle distribution to the back of the eye was clearly increased when the application time was increased from 1.5 min to 3 min. However, there was less of an increase or no significant increase when comparing 3 min to 5 min of application time. The APT for the 3 min and 5 min groups were similar. Without wishing to be bound by theory, since the SCS collapse of the rabbit eye ex vivo starts around 5 minutes, this may inhibit the particle delivery backward. In addition, with increased application time the particles aggregate, which may also inhibit posterior delivery.

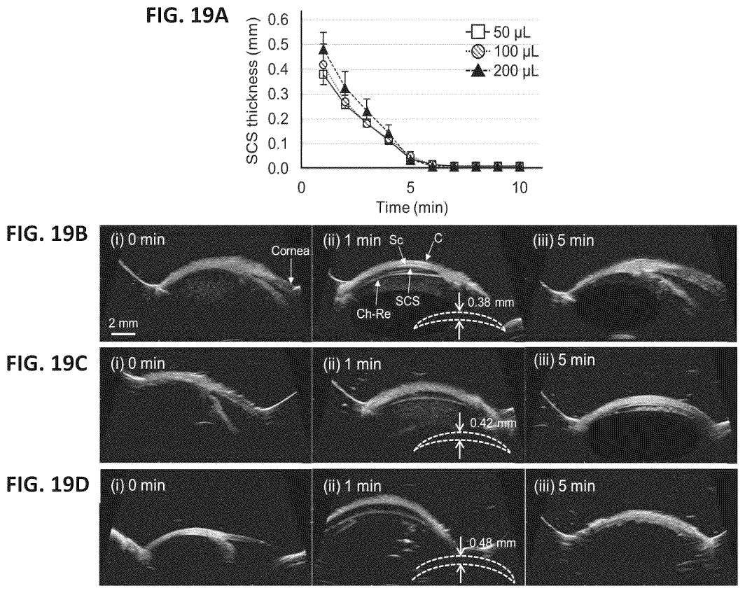

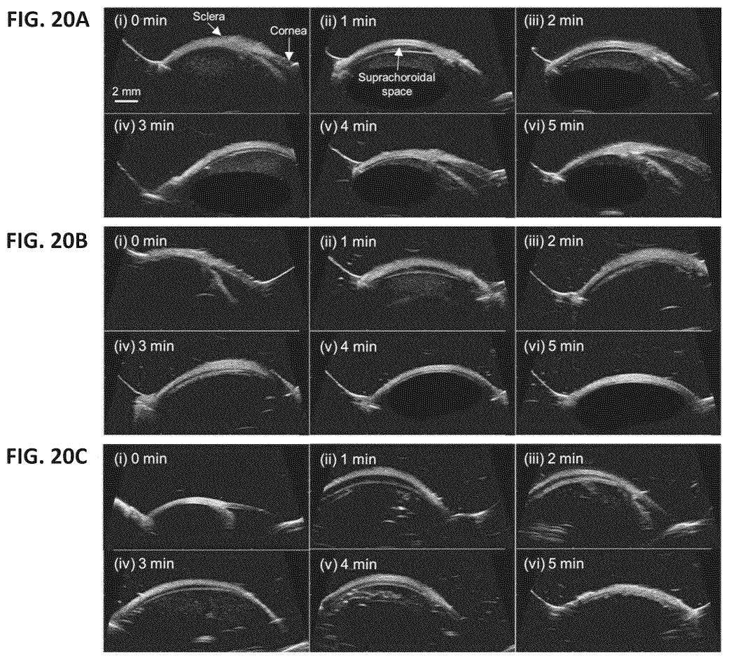

To assess the SCS collapse of the rabbit eye ex vivo, thickness of the SCS was measured by an ultrasound probe. FIG. 19A shows the thickness of the SCS over time with different injection volumes (50, 100, or 200 μL). FIGS. 19B, 19C, and 19D show ultrasound images of the rabbit eyes ex vivo before injection (0 min, (i)), 1 minutes after injection (ii), or 5 minutes after injection (iii) with injection volumes of 50 μL (FIG. 19B) 100 μL (FIGS. 19C) and 200 μL (FIG. 19D). The SCS collapse time was around 5 min regardless of the injection volume. Thus, the SCS collapse of the eye ex vivo affect the particle delivery to the back of the eye at around 5 min. Ultrasound images of the rabbit eyes ex vivo at 0 min, 1 min, 2 min, 3 min, 4 min, and 5 min are also shown in FIGS. 20A-20C for 50 μL (FIGS. 20A), 100 μL (FIG. 20B), and 200 μL (FIG. 20C) injection volumes. The SCS thickness decreased gradually over time.

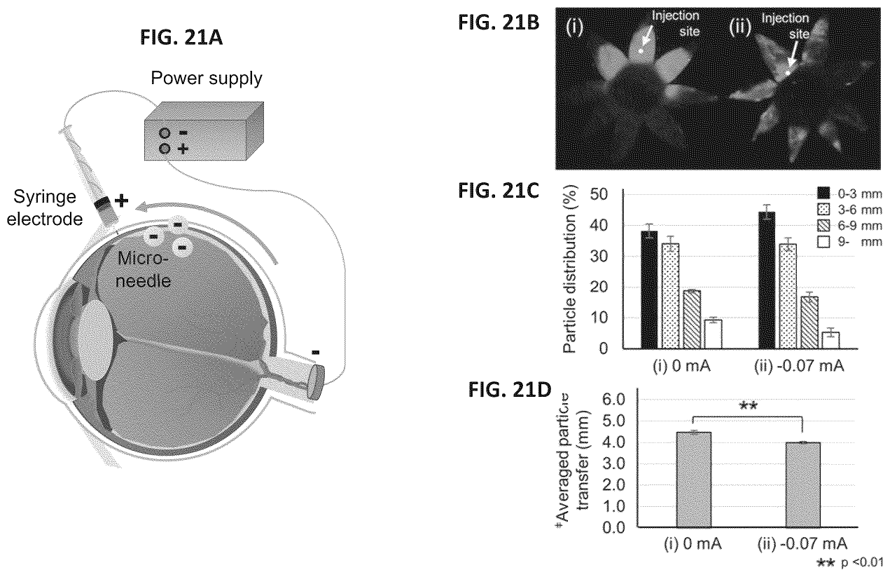

A study of the effect of the iontophoretic direction within the SCS was conducted, with the anode at the injection site and the cathode at the optic nerve. A schematic of the study is provided in FIG. 21A. For this study, particle distribution and transfer was assessed after injection of 100 μL of particle formulation followed by no iontophoresis (i) or reversed direction iontophoresis (−0.07 mA; (ii)). FIG. 21B shows the fluorescence micrographs providing the distributions of the red fluorescent particles in the SCS. The particles stayed around the injection site when the current direction was changed. FIG. 21C provides the particle distribution %, and FIG. 21D shows the APT. Particle distribution focused to the front rather than the back of the eye when the current direction was changed.

Together, the ex vivo studies showed that charged particles injected into the SCS were delivered more preferentially to the anterior or posterior portions of the SCS, depending on the electric current and its polarity. Accordingly, the iontophoresis method provided herein is a viable strategy for targeting drug delivery to specific regions within the SCS.

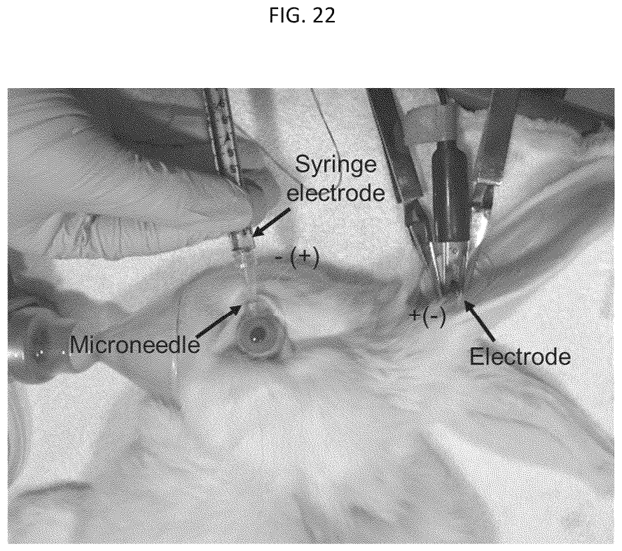

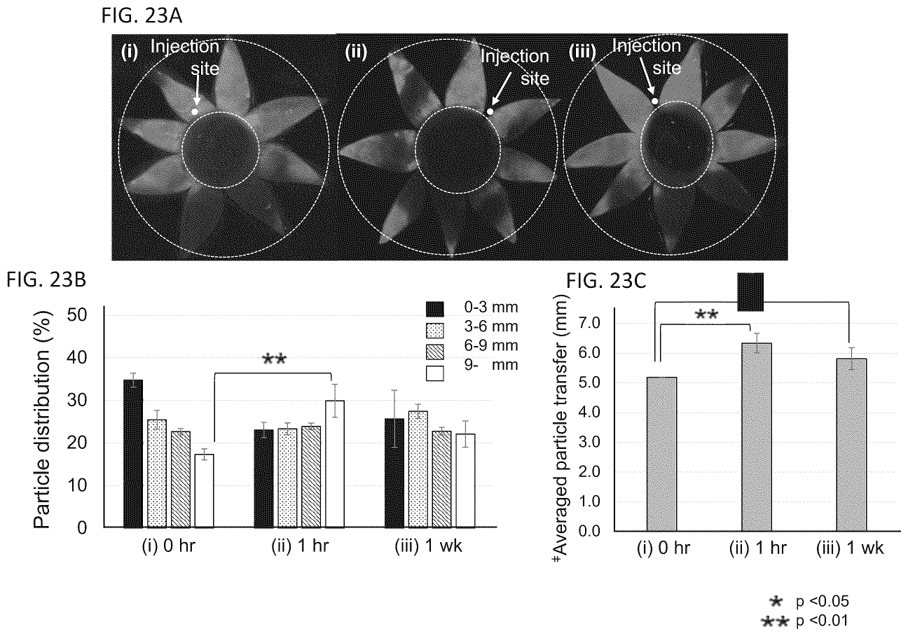

FIG. 22 is a photograph image of an exemplary in vivo delivery using the iontophoretic ocular drug delivery system. FIG. 23A provides fluorescent micrographs showing the distribution of the red-fluorescent particles in the SCS after in vivo injection of a volume of 100 μL red fluorescent particle formulation and iontophoresis of 0.14 mA for 3 minutes. FIG. 23B shows the particle distribution percent at 0 hr, 1 hr, and 1 week after injection. FIG. 23C shows the APT. Although the portion of the particles in the posterior was decreased from (ii) 1 hour to (iii) 1 week, the iontophoresis clearly enhanced the particle delivery to the back of the eye.

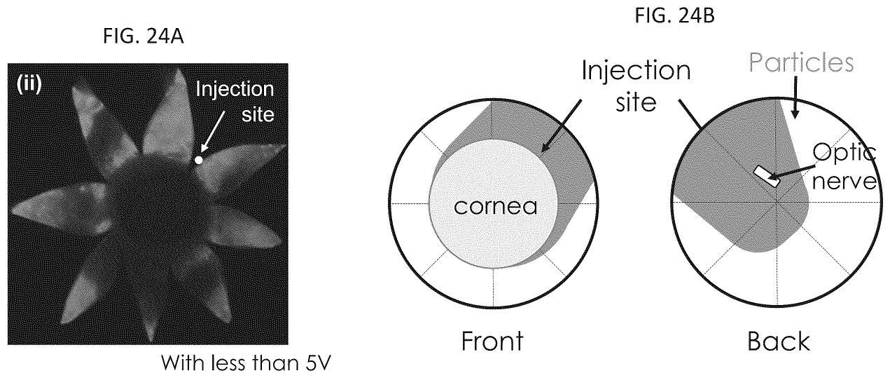

FIG. 24A shows the red particles by fluorescent micrograph 1 hour after in vivo injection of 100 μL red fluorescent particles and iontophoresis using a current of 0.14 mA for 3 minutes (as shown in FIG. 23A(ii)). FIG. 24B is a schematic diagram showing the particle distribution to the back of the eye, if the same eye as shown in FIG. 24A eye were reconstructed. Notably, the discontinued regions in the middle of the petal are not caused from missing tissue. Rather, these regions show the particle delivery around optic nerve derived by iontophoresis. In contrast to the ex vivo studies, the localization of particles in the eye after in vivo administration showed the particles were attracted much more strongly by the iontophoresis.

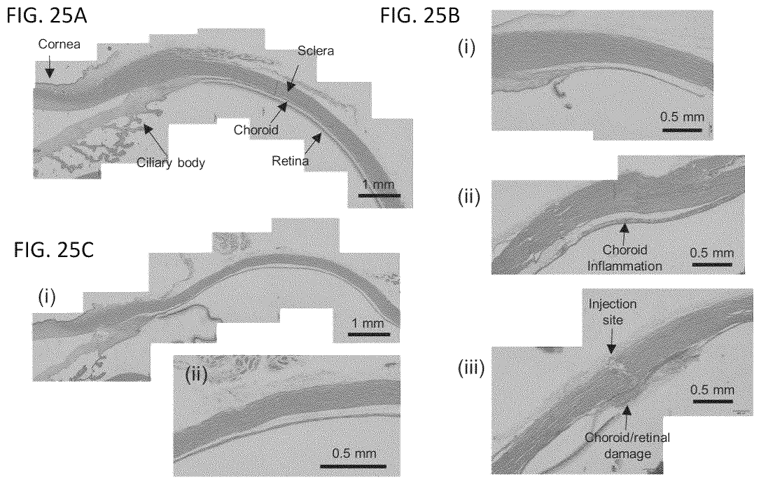

Histology images of the rabbit eye after in vivo injection and iontophoresis were taken to determine if the procedure caused any damage to the eyes. FIG. 25A shows the intact, untreated eye. FIG. 25B shows three images of eyes 1 day after in vivo injection and iontophoresis application. FIG. 25C shows two images of an eye 1 week after in vivo injection and iontophoresis application. Although two of the three eyes after day 1 showed inflammation or choroid/retinal damage near the injection site, there were no symptoms observed in any eyes 1 week after injection. Thus, the study showed that there may be symptoms around 1 day after the iontophoresis procedure, but that eyes are fully recovered within 1 week. There were no other symptoms observed in the rabbits that received the treatment.

### Example 3. Highly Targeted Ocular Drug Delivery by Iontophoresis and Hydrogel Pushing in the Suprachoroidal Space

Suprachoroidal space (SCS) injection using a microneedle has been demonstrated as a means to better target drug delivery specifically to the posterior eye (e.g., macula and optic nerve). Injecting into the SCS, a potential space between the choroid and sclera, allows drug to flow circumferentially at the choroid-sclera interface typically from an anterior injection site to near the limbus with high bioavailability. The safety and tolerability of SCS injection using a microneedle have been shown in clinical trials. Although SCS injection targets drug delivery to choroid and retina, it does not specifically target the posterior pole. Moreover, typical SCS injection volumes (e.g., ≤100 μL in the rabbit eye) are insufficient to flow injected drugs around the macula or optic disk. Greater targeting efficiency within the SCS could provide still better safety and efficacy.

To advance drug targeting to posterior SCS, a previous study (e.g., described herein in Example 2) introduced iontophoresis-mediated drug delivery methods wherein charged drug model particles were delivered to the back of the eye via the SCS. Although over 50% of the drug particle was delivered further posteriorly to the posterior segment of the SCS (i.e., >6 mm posterior to the limbus), there was still around 25% of the drug particles around injection sites (i.e., <3 mm posterior to the limbus).

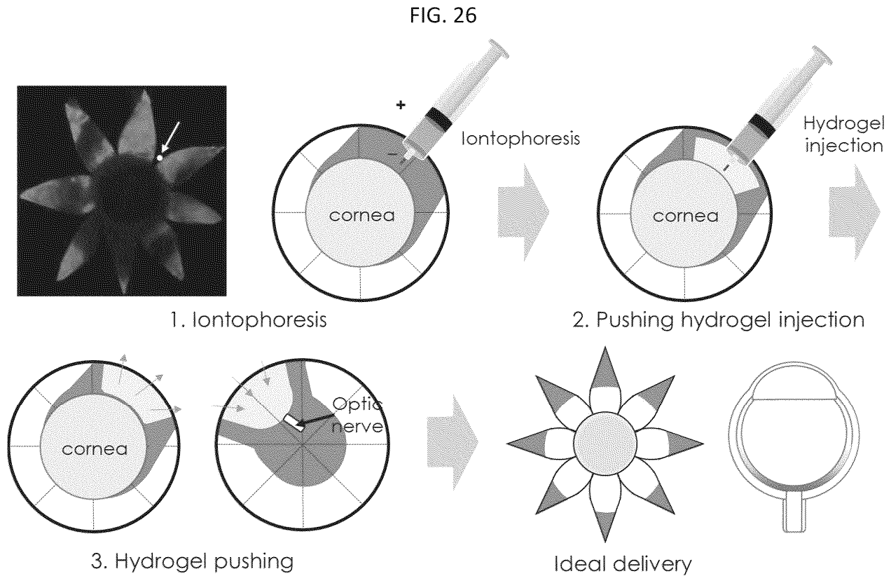

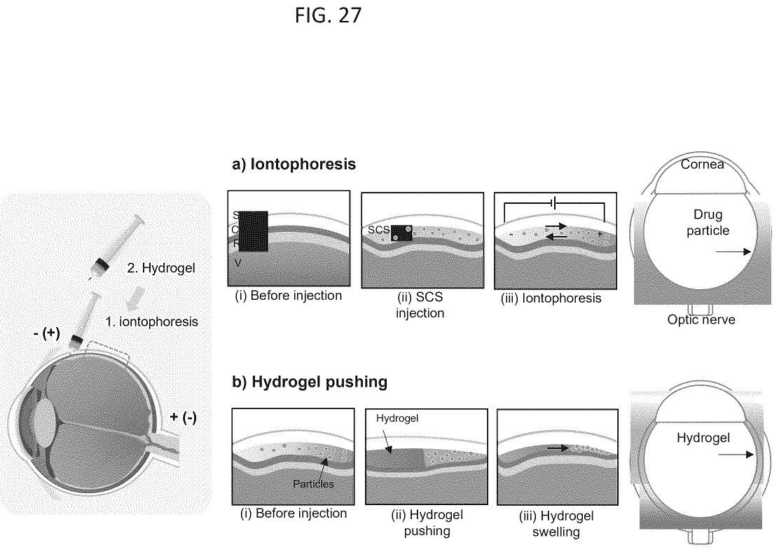

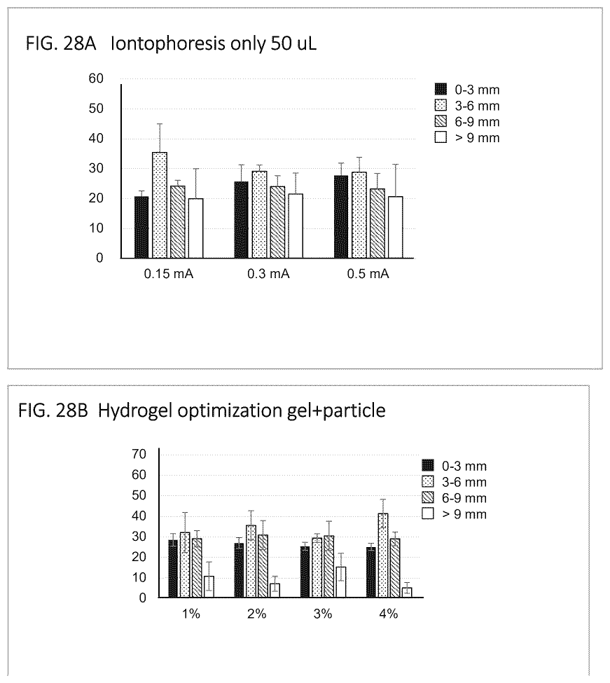

Thus, the present study was conducted to determine if the addition of hyaluronic acid (HA) hydrogel pushing formulation into the SCS, administered right before or after the iontophoresis, would further deliver the drugs around the injection site to the posterior. The HA hydrogel injection not only pushes the particles round the injection site, but also transfer the particles further toward the posterior by hydrogel swelling. Schematic diagrams of the process are provided in FIGS. 26 and 27. FIG. 28A shows the results of different current conditions and injection of 50 μL drug particle formulation (without pushing formulation). Current conditions of 0.15 mA, 0.3 mA, and 0.5 mA were tested, and 0.5 mA was selected for the combination study. FIG. 28B shows the results of different HA formulations for the pushing formulation (tested in the absence of iontophoresis). 1% HA, 2% HA, 3% HA, and 4% HA formulations were tested. 4% HA pushed the particles without mixing, and this concentration was selected for the combination study.

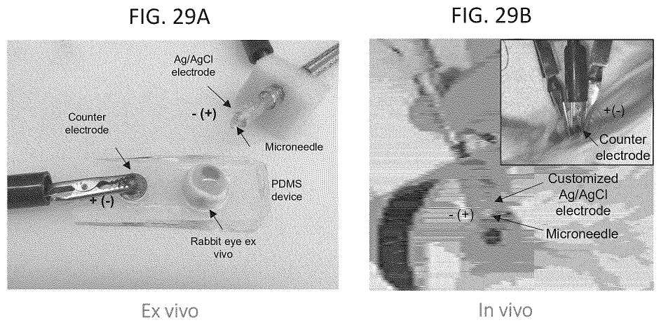

In the combination drug delivery system, a customized Ag/AgCl electrode was located to around a microneedle for iontophoresis. The system was applied to rabbit eyes ex vivo (FIG. 29A) and in vivo (FIG. 29B) to provide an assessment of the delivery and safety of this iontophoretic SCS injection system.

A 30 gauge hollow microneedle, 750 μm in length, was used to inject negatively charged fluorescent particles into the SCS of the rabbit eye ex vivo. A customized ring type Ag/AgCl electrode was attached around a microneedle and the electrode was connected to a DC power supply. A polydimethylsiloxane (PDMS) microdevice was fabricated to apply iontophoresis to the rabbit eye ex vivo and provide similar injection conditions to in vivo injection. Two 0.75 inch diameter holes were made and connected by a single channel, 0.75×0.2×2.2 inch. A rabbit eye and a disk typed Ag/AgCl electrode (0.5 inch diameter) were placed in the holes. The channel was filled with Hank's balanced salt solution (HBSS) buffer. 50 μL of the particle solution was infused into the SCS through the microneedle, simultaneously with application of electric current (0.5 mA) for 3 minutes from the injection site to another Ag/AgCl electrode. To deliver the particles around the injection site (the anterior SCS) to the back of the eye, 50 μL of 4% (w/v) hyaluronic acid hydrogel was infused into the SCS right after the iontophoresis. To confirm the effect of the HA hydrogel swelling, the injected rabbit eyes were incubated in HBSS buffer at 37° C. for 6 h. After the injection and the incubation, particle distribution in the SCS was analyzed.

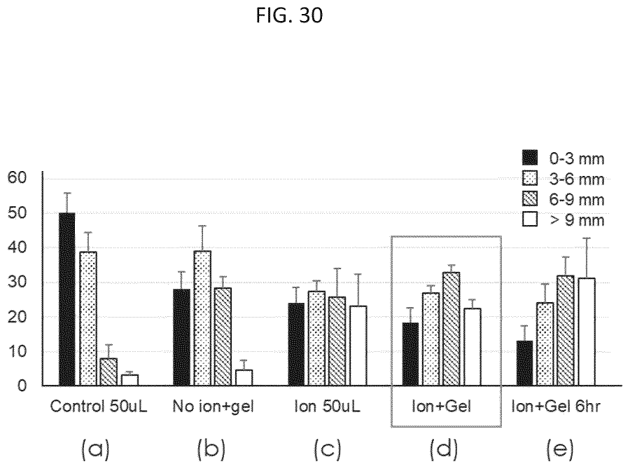

The results of the study are provided in FIG. 30. Particles (50 μL) injected into the SCS of the rabbit eyes ex vivo in the absence of an electric field were distributed more toward the anterior region, ˜50% of the particle formulation was found in the anterior quadrant of the SCS (i.e., <3 mm posterior to the limbus) and only ˜10% of the particle formulation was found in the posterior quadrant of the SCS (i.e., >6 mm posterior to the limbus). The particle injection with hydrogel injection (50 μL), without iontophoresis, delivered ˜28% of particles in the anterior SCS and ˜32% of particles in the posterior SCS. When the iontophoresis was applied only (no hydrogel pushing), ˜24% of particles were delivered to the anterior SCS and ˜49% of particles located in the posterior SCS. Where the iontophoresis was applied first and then hydrogel pushing followed, ˜18% of particles were distributed in the anterior SCS and ˜55% of particles were in the posterior SCS. After 6 h incubation of the eye which was applied iontophoresis first and injected HA hydrogel second, ˜13% of particles were distributed in the anterior SCS and ˜63% of particles in the posterior SCS. Therefore, the study showed that the combination of iontophoresis with hydrogel pushing, including incubation time ex vivo to allow hydrogel swelling, resulted in an increase in the delivery of particles to the further posterior positions in the posterior segment of the eye.

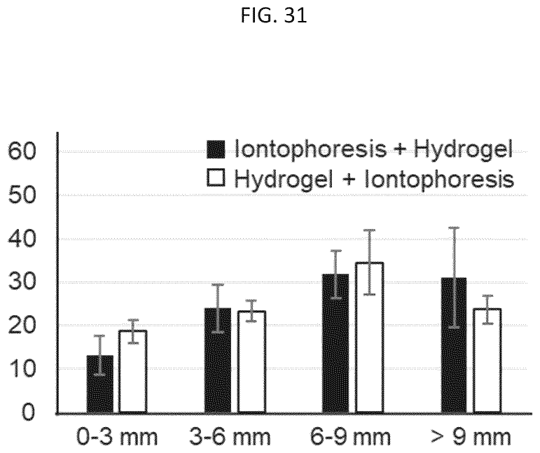

An additional study was conducted to determine if applying iontophoresis after hydrogel pushing, rather than immediately before hydrogel pushing, would increase or decrease particle delivery to the posterior SCS. FIG. 31 shows the comparison of the localization of the particles within the eye when the iontophoresis was applied before or after the administration of the hydrogel pushing formulation. There was no significant difference between the two groups.

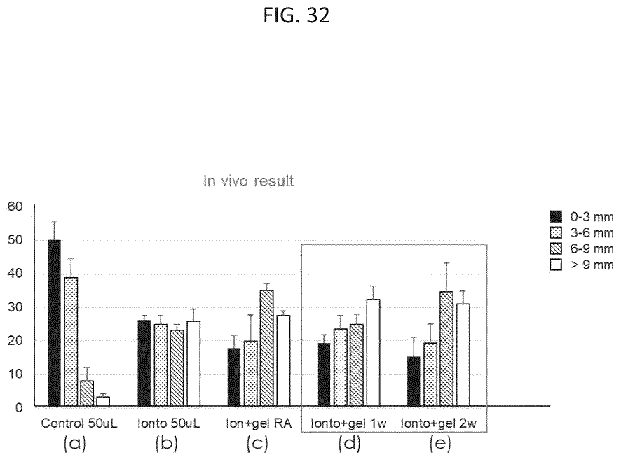

Based on the ex vivo results, SCS injection into the New Zealand White rabbit eyes was tested in vivo. The results are provided in FIG. 32, and correlated with the ex vivo results. Red fluorescent particles were injected to the SCS, then iontophoresis was applied for 3 minutes. Subsequently, the hydrogel formulation was injected at the same injection site. Two weeks after the injection, the particle distribution was analyzed. ˜15% of particles were in the anterior SCS and ˜65% of particles were in the posterior SCS. Thus, the charged particles injected into the SCS were delivered more preferentially to the posterior of the SCS by iontophoresis. HA hydrogel pushing delivered the particles further to the posterior. 2 weeks after the injection, even more particles were delivered to the back compared to the results at 1 week, indicating that hydrogel swelling was still working even an additional week after injection.

Together, the results of the study showed that iontophoresis and hydrogel pushing in the suprachoroidal space can target delivery of particles to the back of the eye, which may be of use for drug delivery of posterior ocular diseases.

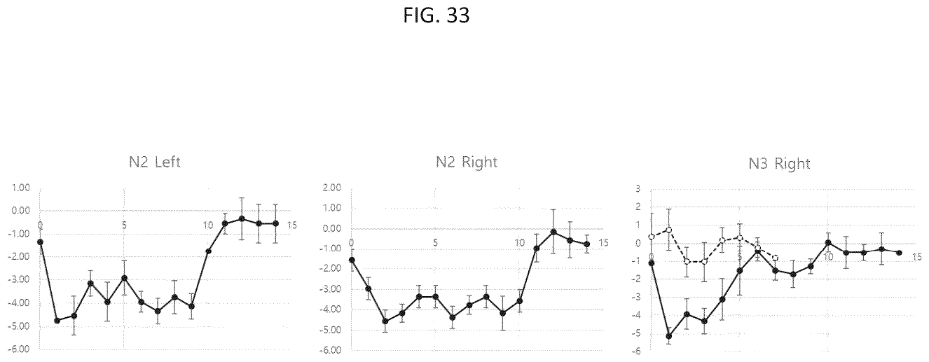

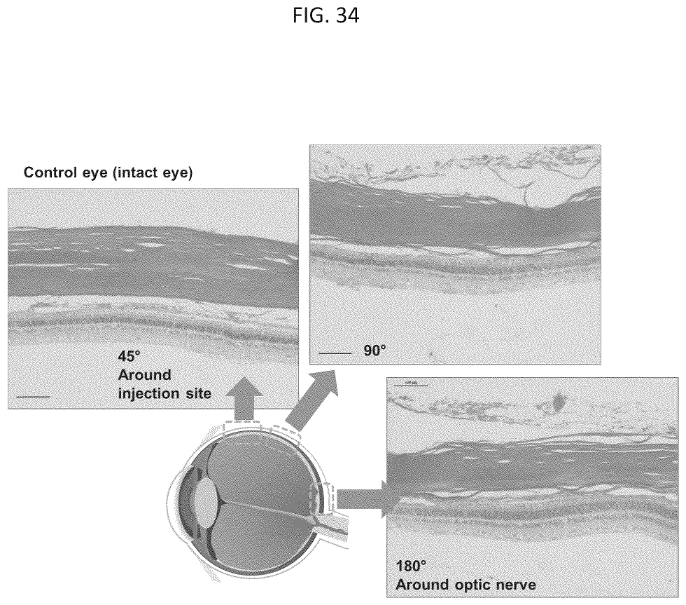

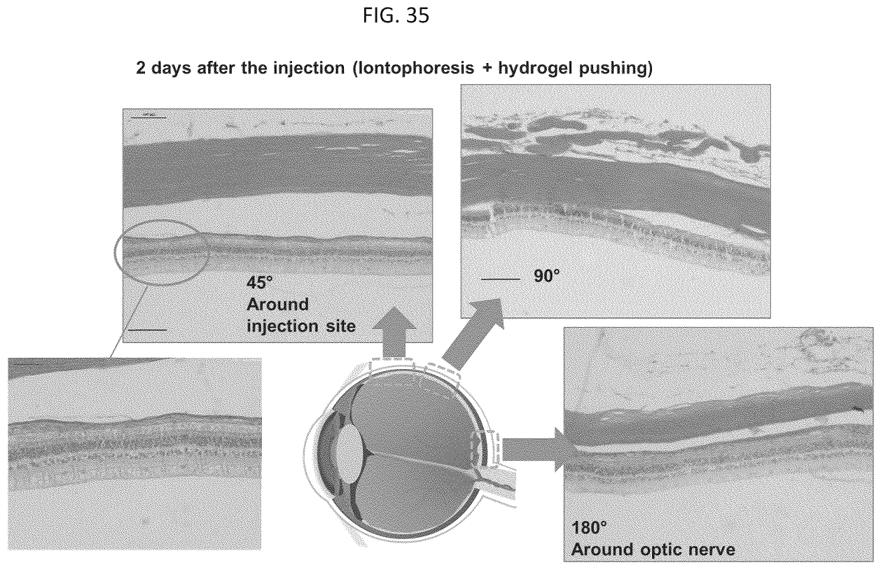

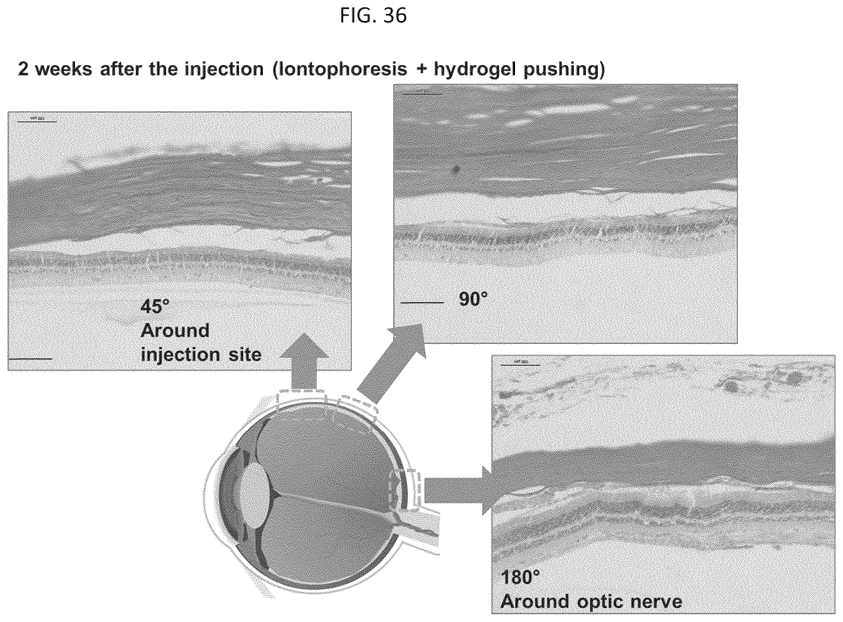

In addition, to confirm the safety of this drug delivery system, intraocular pressure (IOP) was measured after the injection for 2 weeks and the injected rabbit eyes were analyzed in vivo 2 days and 2 weeks after the injection by histology. Although the IOP was lowered about 4 mmHg right after the injection, it recovered around 9 days after the injection (FIG. 33). FIG. 34 shows the histology of a control eye. FIGS. 35 and 36 show the histology of the eyes 2 days or 2 weeks, respectively, after iontophoresis and hydrogel pushing. Although there was a slight inflammatory response 2 days after the injection, there was not significant damage to the retina, choroid, or other areas at 2 days or at 2 weeks after injection. Accordingly, the combination treatment of iontophoresis with hydrogel pushing in SCS drug delivery was safe as well as effective.

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

1. A method for delivery of a drug formulation to a target tissue in the eye of a subject, the method comprising administering to the suprachoroidal space (SCS) of the subject a first formulation and a second formulation, wherein the first formulation comprises an active agent and the second formulation is a pushing formulation.

2. The method of claim 1, wherein the first formulation and the second formulation are administered to the SCS using a microneedle.

3. The method of claim 1, wherein the first formulation and the second formulation are administered to the SCS using a device containing both the first and second formulation.

4. The method of claim 1, wherein the first formulation is administered to the SCS prior to administration to the SCS of the pushing formulation.

5. The method of claim 1, wherein the first formulation has a lower viscosity than the pushing formulation.

6. The method of claim 1, wherein the pushing formulation comprises a gel.

7. The method of claim 6, wherein the pushing formulation comprises hyaluronic acid (HA).

8. The method of claim 7, wherein the pushing formulation comprises about 4% (w/v) HA.

9. The method of claim 1, wherein the pushing formulation is a high-salt formulation.

10. The method of claim 9, wherein the pushing formulation comprises 9% sodium chloride.

11. The method of claim 1, wherein the first formulation comprises particles.

12. The method of claim 11, wherein the particles are microparticles or nanoparticles.

13. The method of claim 1, wherein the first formulation further comprises HA.

14. The method of claim 13, wherein the first formulation comprises about 1% (w/v) HA.

15. The method of claim 1, wherein the first formulation comprises about 1% (w/v) HA and the second formulation comprises about 4% (w/v) HA.

16. The method of claim 1, wherein the target tissue of the eye is selected from the group consisting of the posterior pole, optic nerve, choroid, retina, vitreous humor, macula, iris, or ciliary body.

17. The method of claim 2, wherein the microneedle is a hollow microneedle.

18. The method of claim 17, wherein the hollow microneedle is between about 600 μm and about 1200 μm in length.

19. The method of claim 18, wherein the hollow microneedle is about 900 μm in length.

20. The method of claim 18, wherein the hollow microneedle is about 1100 μm in length.

21. The method of claim 1, wherein the method results in localization of at least 30% of the first formulation to a location in the eye that is at least about 6 mm posterior to the limbus.

22. The method of claim 1, wherein the method results in localization of at least about 50% of the first formulation to a location in the eye that is at least about 6 mm posterior to the limbus.

23. The method of claim 1, wherein the method results in localization of at least about 60% of the first formulation to a location in the eye that is at least about 3 mm posterior to the limbus.

24. The method of claim 1, wherein the pushing formulation swells over time and pushes the first formulation further posteriorly.

25. The method of claim 24, wherein the pushing formulation swells over time for at least 1 hour after injection.

26. The method of claim 24, wherein the formulation remains further posteriorly for at least 2 days.

27. The method of claim 24, wherein the formulation remains further posteriorly for at least 2 weeks.

28. A device comprising a microneedle and a syringe, wherein the device comprises a first formulation and a second formulation, wherein the first formulation comprises an active agent and the second formulation is a pushing formulation.

29. The device of claim 28, wherein the syringe is coupled to a microneedle.

30. The device of claim 28, wherein the first formulation has a lower viscosity than the second formulation.

31. The device of claim 28, wherein the first formulation and the second formulation are substantially separate.

32. The device of claim 28, wherein the pushing formulation comprises a gel.

33. The syringe of claim 32, wherein the gel is hyaluronic acid (HA).

34. The syringe of claim 33, wherein the second formulation comprises about 4% (w/v) HA.

35. The device of claim 28, wherein the pushing formulation is a high salt formulation.

36. The syringe of claim 35, wherein the pushing formulation comprises 9% sodium chloride.

37. The device of claim 28, wherein the first formulation comprises particles.

38. The device of claim 28, wherein the first formulation comprises about 1% (w/v) HA and the second formulation comprises about 4% (w/v) HA.

39. A method for treating an ocular disease or disorder in a subject in need thereof, the method comprising administering to the suprachoroidal space (SCS) of the subject a first formulation and a second formulation, wherein the first formulation comprises an active agent and the second formulation is a pushing formulation.

40. The method of claim 39, wherein the first formulation and the second formulation are administered to the SCS using a microneedle.

41. The method of claim 39, wherein the first formulation and the second formulation are administered to the SCS using a device containing both the first and second formulation

42. The method of claim 39, wherein the first formulation is administered to the SCS first and is followed by the pushing formulation.

43. The method of claim 39, wherein the first formulation has a lower viscosity than the pushing formulation.

44. The method of claim 39, wherein the pushing formulation comprises a gel.

45. The method of claim 44, wherein the pushing formulation comprises hyaluronic acid (HA).

46. The method of claim 45, wherein the pushing formulation comprises about 4% (w/v) HA.

47. The method of claim 39, wherein the pushing formulation is a high-salt formulation.

48. The method of claim 47, wherein the pushing formulation comprises 9% sodium chloride.

49. The method of claim 39, wherein the first formulation comprises particles.

50. The method of claim 49, wherein the particles are microparticles or nanoparticles.

51. The method of claim 39, wherein the first formulation further comprises HA.

52. The method of claim 51, wherein the first formulation comprises about 1% (w/v) HA.

53. The method of claim 39, wherein the first formulation comprises about 1% (w/v) HA and the second formulation comprises about 4% (w/v) HA.

54. The method of claim 39, wherein the disease or disorder is selected from the group consisting of uveitis, retinal vein occlusion, diabetic retinopathy, diabetic macula edema, and age-related macular degeneration.

55. The method of claim 39, wherein the pushing formulation pushes the first formulation further posteriorly in the posterior segment of the eye.

56. The method of claim 39, wherein the disease or disorder is glaucoma, and wherein the pushing formulation pushes the first formulation to the ciliary body.

57. The method of claim 39, wherein the microneedle is a hollow microneedle.

58. The method of claim 57, wherein the hollow microneedle is between about 600 μm and about 1200 μm in length.

59. The method of claim 58, wherein the hollow microneedle is about 900 μm in length.

60. The method of claim 58, wherein the hollow microneedle is about 1100 μm in length.

61. The method of claim 39, wherein the method results in localization of at least 30% of the first formulation to a location in the eye that is at least about 6 mm posterior to the limbus.

62. The method of claim 39, wherein the method results in localization of at least about 50% of the first formulation to a location in the eye that is at least about 6 mm posterior to the limbus.

63. The method of claim 39, wherein the method results in localization of at least about 60% of the first formulation to a location in the eye that is at least about 3 mm posterior to the limbus.

64. A method for delivery of a drug formulation to a target tissue in the eye of a subject, the method comprising administering to the suprachoroidal space (SCS) of the subject a first formulation and a second formulation, wherein the first formulation is a pushing formulation and the second formulation comprises an active agent.

65. A first formulation and a second formulation for use in a method of delivering a drug formulation to a target tissue in the eye of a subject, wherein the first formulation and second formulation are delivered to the suprachoroidal space (SCS) of the eye of the subject, wherein the first formulation comprises an active agent and the second formulation is a pushing formulation.

66. A first formulation and a second formulation for use in a method of treating a posterior ocular disease or disorder in a subject in need thereof, wherein the first formulation and second formulation are delivered to the suprachoroidal space (SCS) of the eye of the subject, wherein the first formulation comprises an active agent and the second formulation is a pushing formulation.

67. A method for delivery of a drug formulation to a target tissue in the eye of a subject, the method comprising administering to the suprachoroidal space (SCS) of the subject a formulation comprising an active agent and further comprising applying iontophoresis to the eye of the subject to localize the active agent to a target tissue in the eye.

68. The method of claim 67, wherein the formulation comprising the active agent is administered to the SCS using a hollow microneedle.

69. The method of claim 67, wherein the formulation comprising the active agent is administered to the SCS prior to the application of iontophoresis to the eye.

70. The method of claim 67, wherein the formulation comprising the active agent is administered to the SCS concurrently with the application of iontophoresis to the eye.

71. The method of claim 67, wherein the active agent has a net negative charge.

72. The method of claim 67, wherein the active agent comprises drug particles or drug encapsulated in particles, and wherein the particles have a net negative charge.

73. The method of claim 67, wherein the iontophoresis applied to the eye comprises a current with an absolute value of about 0.05 mA to about 0.8 mA.

74. The method of claim 73, wherein the iontophoresis applied to the eye comprises a current with an absolute value of about 0.1 mA to about 0.2 mA.

75. The method of claim 73, wherein a positive current is used with an active agent having a positive charge.

76. The method of claim 73, wherein a negative current is used with an active agent having a positive charge. t−0.07 mA.

77. The method of claim 73, wherein a negative current is used with an active agent having a negative charge.

78. the method of claim 73, wherein a positive current is used with an active agent having a positive charge.

79. The method of claim 67, wherein the iontophoresis is applied to the eye for about 1 minute to about 10 minutes.

80. The method of claim 79, wherein the iontophoresis is applied to the eye for about 3 minutes.

81. A method for delivery of a drug formulation to a target tissue in the eye of a subject, the method comprising
(i) administering to the suprachoroidal space (SCS) of the eye of the subject a first formulation and a second formulation, wherein the first formulation comprises an active agent and the second formulation is a pushing formulation; and
(ii) applying iontophoresis to the eye of the subject to localize the active agent to a target tissue in the eye.

82. The method of claim 81, wherein the first and second formulations are each administered to the SCS via a hollow microneedle.

83. The method of claim 82, wherein the first and second formulations are contained in the same syringe, wherein the syringe is coupled to the microneedle.

84. The method of claim 81, wherein the first and second formulations are contained in separate microneedles.

85. The method of claim 81, wherein the pushing formulation enters the SCS after the first formulation.

86. The method of claim 81, wherein the first formulation has a lower viscosity than the pushing formulation.

87. The method of claim 81, wherein the pushing formulation comprises about 4% hyaluronic acid (HA).

88. The method of claim 87, wherein the pushing formulation further comprises about 9% sodium chloride.

89. The method of claim 81, wherein the first formulation comprises about 1% HA.

90. The method of claim 81, wherein the active agent has a net negative charge.

91. The method of claim 81, wherein the first formulation comprises particles having a net negative charge.

92. The method of claim 81, wherein the iontophoresis is applied to the eye after administration of the first formulation and before administration of the second formulation.

93. The method of claim 81, wherein the iontophoresis is applied to the eye after the first and second formulations are administered to the SCS of the eye.

94. The method of claim 81, wherein the iontophoresis applied to the eye comprises a current of about 0.14 mA.

95. The method of claim 94, wherein the iontophoresis is applied to the eye for about 3 minutes.

96. The method of claim 81, wherein the method pushes the first formulation further posteriorly in the posterior segment of the eye.

97. The method of claim 81, wherein the method results in localization of at least 30% of the first formulation to a location in the eye that is at least about 6 mm posterior to the limbus.

98. The method of claim 81, wherein the method results in localization of at least about 50% of the first formulation to a location in the eye that is at least about 6 mm posterior to the limbus.

99. The method of claim 81, wherein the iontophoresis is applied to the eye prior to the administration of the second formulation.

100. The method of claim 81, wherein the iontophoresis is applied to the eye after administration of the second formulation.

101. The method of claim 81, wherein the method comprises the following sequential steps:
(i) administering to the SCS of the eye of the subject a first formulation comprising an active agent;
(ii) applying iontophoresis to the eye of the subject; and
(iii) administering to the SCS of the eye of the subject a second formulation, wherein the second formulation is a pushing formulation.

102. A method treating an ocular disease or disorder in a subject in need thereof, the method comprising
(i) administering to the suprachoroidal space (SCS) of the eye of the subject a first formulation and a second formulation, wherein the first formulation comprises an active agent and the second formulation is a pushing formulation; and
(ii) applying iontophoresis to the eye of the subject to localize the active agent to a target tissue in the eye.

103. The method of claim 102, wherein the first and second formulations are administered to the SCS via a hollow microneedle.

104. The method of claim 103, wherein the first and second formulations are contained in the same syringe, wherein the syringe is coupled to the microneedle.

105. The method of claim 102, wherein the pushing formulation enters the SCS after the first formulation.

106. The method of claim 102, wherein the first formulation has a lower viscosity than the pushing formulation.

107. The method of claim 102, wherein the pushing formulation comprises about 4% hyaluronic acid (HA).

108. The method of claim 107, wherein the pushing formulation further comprises about 9% sodium chloride.

109. The method of claim 102, wherein the first formulation comprises about 1% HA.

110. The method of claim 102, wherein the active agent has a net negative charge.

111. The method of claim 102, wherein the first formulation comprises particles having a net negative charge.

112. The method of claim 102, wherein the iontophoresis is applied to the eye after the first and second formulations are administered to the SCS of the eye.

113. The method of claim 102, wherein the iontophoresis applied to the eye comprises a current of about 0.14 mA.

114. The method of claim 102, wherein the iontophoresis is applied to the eye for about 3 minutes.

115. The method of claim 102, wherein the disease or disorder is a posterior ocular disorder.

116. A first formulation, a second formulation, and iontophoresis for use in a method of treating a posterior ocular disease or disorder in a subject in need thereof, wherein the first formulation and second formulation are delivered to the suprachoroidal space (SCS) of the eye of the subject, wherein the first formulation comprises an active agent and the second formulation is a pushing formulation, and wherein the iontophoresis is applied to the eye of the subject to target the active agent to a target tissue in the eye.
